(19) United States
(12) Patent Application Publication
(10) Pub. No.: US 2006/0134067 A1
(43) Pub. Date: Jun. 22, 2006

(54) Loading of cells with antigens by electroporation

(75) Inventors: Linda N. Liu, Clarksville, MD (US); Jonathan M. Weiss, Rockville, MD (US)

Correspondence Address: FULBRIGHT & JAWORSKI L.L.P., 600 CONGRESS AVE., SUITE 2400, AUSTIN, TX, 78701, US

(73) Assignee: Maxcyte, Inc.

(21) Appl. No.: 11/219,307

(22) Filed: Sep. 2, 2005

Related U.S. Application Data
(63) Continuation-in-part of application No. 10/781,440, filed on Feb. 18, 2004.
(60) Provisional application No. 60/448,670, filed on Feb. 18, 2003.

(51) Int. Cl. C12N 15/86; C12N 15/87; A61K 48/00; C12N 15/74

(52) U.S. Cl. 424/93.2; 435/459; 435/456; 435/471

(57) ABSTRACT

Methods for loading an antigen-presenting cell with one ore more antigens are disclosed. Methods for the treatment and prevention of a disease in a subject using an antigen-presenting cell that has been electroporated with a composition of one or more antigens. Composition of one or more antigens comprises one or more antigens of a hyperproliferative cell, a microorganism or a microorganism-infected cell are also disclosed. In addition, compositions of antigen-presenting cells that have been loaded with one or more antigens of a hyperproliferative cell, a microorganism-infected cell or a microorganism using electroporation are disclosed.

### CROSS-REFERENCE TO RELATED APPLICATIONS

This application is a continuation-in-part of U.S. application Ser. No. 10/781,440 filed Feb. 18, 2004, which claims priority to U.S. Provisional Application No. 60/448,670 filed Feb. 18, 2003, both of which are incorporated herein in their entirety.

### TECHNICAL FIELD

The present invention relates generally to the fields of cell biology, microbiology, cancer biology, and immunology. More particularly, it concerns methods for loading an antigen-presenting cell with one or more antigens involving electroporation, and compositions of loaded antigen-presenting cells. Antigens used in the present invention include a hyperproliferative cell, a microorganism-infected cell, or a microorganism. It also concerns methods for the treatment and prevention of a disease, such as cancer or any other infectious disease, in a subject using antigen-presenting cells that have been loaded with one or more antigens of a hyperproliferative cell, a microorganism-infected cell, or a microorganism.

### BACKGROUND OF THE INVENTION

Immune response to foreign antigens and TAAs typically begins with uptake of the antigen by dendritic cells, which are the most efficient antigen presenting cells of the immune system. Dendritic cells (DCs) represent the body's most efficient antigen presenting cell type (APC), being capable of phagocytosing foreign antigens and presenting them to both naïve and memory T cells (Van Schooten et al., 1997; Mellman and Steinman, 2001). Other types of APC include macrophages and B cells.

DCs normally take up antigens by micropinocytosis and/or phagocytosis, after which they process the antigens intracellularly and then present the antigens to T cells of the immune system. While this process normally takes place within the body, it is possible to remove DCs from the body, culture them in vitro, provide antigens to these cultured dendritic cells in vitro, and then return the cells to the body where they can interact with T cells to elicit an augmented immune response against that antigen of interest. This process of providing these antigens to DCs is generally referred to as “pulsing” or co-culturing and is generally effected by simply adding the antigens to the DCs and allowing the DCs to micropinocytose and/or phagacytose the antigens. Mixture of DCs ex vivo with the antigen increases opportunities for DCs micropinocytosis and/or phagocytosis of antigen and overcomes lower efficiency of in vivo setting. Use of such co-culturing to provide tumor antigens to DCs has been described (Schnurr et al., 2002; Herr et al., 2000; Geiger et al., 2001).

Purified tumor-associated antigens have been characterized for some tumors and have been used as cancer vaccines with some success (Höltl et al., 2002; Asavaroengchai et al., 2002). However, identification of TAAs has been limited. In addition, the use of purified and characterized TAAs may not be feasible for all cancers. In situations in which TAAs are known and can be purified and loaded into DCs, a more efficient method for loading would reduce the amount of antigen needed. Accordingly, there is great interest in identification of a method that will allow the TAAs to be more efficiently presented to APCs.

Electroporation has been described as a means to introduce non-permeant molecules into living cells (reviewed in Mir, 2000). At the level of the entire cell, the consequences of cell exposure to the electric pulses are not completely understood. In the presence of the external electric field, a change in the transmembrane potential difference is believed to be generated (Neumann et al., 1999; Weaver and Chizmadzhev, 1996; Kakorin et al., 1996). This electric field is superimposed upon the resting transmembrane potential difference and it may be calculated from the Maxwell's equations, providing a few approximations are made (very reduced thickness of the cell membrane, null membrane conductivity, etc.) (Mir, 2000). These changes in the transmembrane potential difference have been experimentally observed (Hibino et al., 1993; Gabriel and Teissié, 1999). Analytically, the effects of the exposure of cells to electric pulses are well described in the case of isolated cells in suspension (Kotnik et al., 1998).

At the molecular level of analysis, the explanation of the phenomena occurring at the cell membrane level is hypothetical. It is assumed that above a threshold value of the net transmembrane potential, the changes occurring in membrane structure will be enough as to render that membrane permeable to otherwise non-permeable molecules of given physicochemical characteristics (molecular mass, radius, etc.) (see Mir, 2000).

Electroporation is most commonly used to introduce DNA (Knutson and Yee, 1987) and RNA (Van Meirvenne et al., 2002; Van Tendeloo et al., 2001) into cells. It has also been described as a possible means of introducing other macromolecules into the cytoplasm of living cells, including antigen-presenting cells (Zhou et al., 1995; Harding, 1992; Chen et al., 1993; Li et al., 1994; Kim et al., 2002).

However, methods are lacking for efficient use of electroporation in the treatment of cancer, other hyperproliferative diseases and other diseases caused by microorganisms, such as infectious diseases. In particular, previous studies have not described methods for use of electroporation to develop immune responses to cancer antigens or other pathogenic antigens, particular with respect to uncharacterized antigens. Development of such techniques would represent a significant advance in cancer therapeutics and other vaccines.

### BRIEF SUMMARY OF THE INVENTION

Accordingly, one of the objects of the present invention is to provide a novel method for loading an antigen-presenting cell (APC) with one or more antigens, comprising: (a) preparing a mixture comprising antigen-presenting cells and an antigen composition comprising one or more antigens; and (b) electroporating the mixture in a manner sufficient to load the antigen composition into the antigen-presenting cells. Although any method of electroporation is contemplated by the present invention, in certain embodiments, electroporating the mixture comprises use of an electroporation device as described in U.S. Publication No. US20030073238A1, which is incorporated herein in its entirety. The methods for loading an APC of the present invention contemplate use of any type of APC. In a certain embodiment, the APC is a dendritic cell. The antigen composition can include one or more of any type of antigen, for example one or more antigens from a hyperproliferative cell, a microorganism-infected cell or a microorganism. More particularly, antigens from a hyperproliferative cell can be tumor-associated antigens or tumor-restricted antigens. The antigen composition may be a lysate. The lysate can be prepared by any method known to one of skill in the art. For example, detergent or non-detergent treatments can be used to prepare a lysate. In certain embodiments, the lysate is prepared using a non-detergent treatment selected from the group consisting of freeze-thaw methods, sonication methods, high pressure extrusion methods, solid shear methods, liquid shear methods, and hypotonic/hypertonic methods. More particularly, the cell and/or a microorganism is subjected to at least one freeze-thaw cycle as part of the method to prepare a lysate. In other embodiments, the lysate is prepared by subjecting the cell and/or microorganism to at least about 2-5 freeze-thaw cycles. In still other embodiments, the lysate is centrifuged following said at least one freeze-thaw cycle. In certain aspects of the invention, the ratio of antigen presenting cells to cells represented in the lysate is greater than 1 antigen presenting cell per lysed cell. Preferably the ratio of antigen presenting cells to cells represented in the lysate is between about 2:1 to 150:1, 2:1 to 100:1, 2:1 to 50:1, or 2:1 to 20:1. More preferably the ratio of antigen presenting cells to cells represented in the lysate is between about 5:1 to 150:1, 5:1 to 100:1, 5:1 to 50:1, or 5:1 to 20:1. In certain aspects of the invention the ratio of antigen presenting cells to cells represented in the lysate is about 10:1. In certain aspects of the invention the protein concentration of the lysate is less than 5000 μg/ml. In some embodiments of the invention the protein concentration of the lysate is between about 5 μg/ml to 5000 g/ml, 5 μg/ml to 2500 μg/ml, 5 μg/ml to 1000 μg/ml, or 5 μg/ml to 750 μg/ml. More preferably the protein concentration is between about 50 μg/ml to 750 μg/ml or between about 250 μg/ml to 750 μg/ml. In yet other aspects of the invention the protein concentration of the lysate is about 50 μg/ml or less. In certain aspects of the invention less than about 100, 90, 80, 70, 60, 50, 40, 30, 20, 10, 9, 8, 5, 4, 3, 2, or 1 μg of lysate protein is used per 1×10⁶ antigen presenting cells. Preferably between about 1 μg to 100 μg of lysate protein is used per 1×10⁶ antigen presenting cells. More preferably between about 1 μg to 50 μg, 1 μg to 40 μg, 1 μg to 20 μg, or 1 μg to 10 μg of lysate protein is used per 1×10⁶ antigen presenting cells.

In a certain embodiment, the lysate is a tumor-cell lysate. The tumor cell lysate can be composed of either benign cells, cancer cells, autologous tumor cells of a subject, allogeneic tumor cells, or a mixture of these cells Although cells of any cancer type are contemplated by the present invention, particular examples of cancer cells include breast cancer cells, lung cancer cells, prostate cancer cells, ovarian cancer cells, brain cancer cells, liver cancer cells, cervical cancer cells, colon cancer cells, renal cancer cells, skin cancer cells, head & neck cancer cells, bone cancer cells, esophageal cancer cells, bladder cancer cells, uterine cancer cells, lymphatic cancer cells, stomach cancer cells, pancreatic cancer cells, testicular cancer cells, or leukemia cells. In some embodiments the present invention provides a method for loading an antigen-presenting cell with one or more antigens, comprising: preparing a mixture comprising antigen-presenting cells and an antigen composition comprising a lysate having one or more antigens of a hyperproliferative cell, a microorganism-infected cell or a microorganism, wherein the number of antigen-presenting cells is greater than the number of cells represented in the lysate; and electroporating the mixture in a manner sufficient to load the one or more antigens into the antigen-presenting cells.

In further embodiments, the lysate is a microorganism-infected cell lysate or a lysate of a microorganism. The microorganism-infected cell lysate is prepared from any cell type that is infected with a microorganism, such as bacteria, viruses, parasites, protozoa, fungi, or any other pathogenic particle.

Another object of the present invention is to provide a novel method for loading an APC with one or more antigens, comprising: (a) preparing a mixture comprising antigen-presenting cells and an antigen composition comprising one or more antigens; and (b) electroporating the mixture in a manner sufficient to load one or more of the antigens into the APC. An antigen as used herein may include any antigen that is not native to the APC or the organism from which the APC is obtained. Any cell and/or microorganism can be the source of the antigen.

Another object of the present invention is to provide methods of treating a subject for a disease, comprising (a) loading an antigen-presenting cell with one or more antigens using any of the methods that have been described above; (b) preparing a composition of said antigen-presenting cell; and (c) administering to a subject in need thereof an effective amount of the composition. In certain embodiments, the one or more antigens include antigens from a /hyperproliferative cell, a microorganism and/or a microorganism-infected cell. In a certain embodiment, the method of loading of the antigen-presenting cell with one or more antigens further comprises use of an electroporation device as described in U.S. Publication No. US20030073238A1. The disease is a hyperproliferative disease or an infectious disease. In another certain embodiment, the method of treating the subject for a disease further comprises culturing the antigen-presenting cell.

The methods of treating a subject for a disease, may involve use of antigens that are substantially purified or use of antigens that are not substantially purified. One of skill in the art would be familiar with the technique to purify antigens. In a certain embodiment, the subject to be treated for a disease is a mammal. In a certain embodiment, the subject is a human. The human can be any human with a disease. In a certain embodiment, the disease is a hyperproliferative disease, for example, the hyperproliferative disease can be a tumor. The tumor can be benign or the tumor can be a cancer. For example, the cancer can be breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer (e.g., Hodgkin lymphoma or non-Hodgkin lymphoma), stomach cancer, pancreatic cancer, testicular cancer, melanoma, myeloma, or leukemia. The lung cancer may be, for example, small cell lung carcinoma (SCLC) or non-small cell lung carcinoma (NSCLC). The leukemia may be, for example, acute myelogenous leukemia (AML), chronic myelogenous leukemia (CML), acute lymphocytic leukemia (ALL), or chronic lymphocytic leukemia (CLL). The subject to be treated can be a subject who is undergoing secondary anti-hyperplastic therapy. For example, the secondary anti-hyperplastic therapy is chemotherapy, radiotherapy, immunotherapy, phototherapy, cryotherapy, toxin therapy, hormonal therapy, or surgery.

Any method of delivery of the composition is contemplated by the present invention. One of skill in the art would be familiar with methods of delivery. For example, the composition can be delivered systemically, intravascularly, intradermally, subcutaneously, or locally to a tumor mass. Use of any antigen-presenting cell is contemplated by the present invention. However, in certain embodiments, the antigen-presenting cells are dendritic cells. The method of treating a subject can further involve the step of culturing the antigen-presenting cells following the loading of the antigen-presenting cells. In still other embodiments, the method of treating a subject further involves measuring the immune response of the antigen-presenting cells following loading of the antigen-presenting cells. The immune response may be monitored in vitro by ELISPOT, ELISA, PCR, tumor cell killing, or by any method known to one of skill in the art. Quantification of the immune response may be performed by measurement of tumor sizes, and, in the case of certain tumor models, counting the number of metastases.

It is another object of the present invention to provide methods of preventing the development of a disease in a subject, involving: (a) loading an antigen-presenting cell with one or more antigens; (b) preparing a composition of said antigen-presenting cell; and (c) contacting a subject in need thereof with an effective amount of said composition. In a certain embodiment, loading the antigen-presenting cell comprises use of an electroporation device as described in U.S. Publication No. US20030073238A1. In certain embodiments, the subject is a mammal or a human. In other embodiments, the human is a patient with a history of a disease, for example hyperproliferative disease. Any disease is contemplated by the present invention. For instance, the hyperproliferative disease can be a benign tumor or a cancer. For example, the cancer is breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, or leukemia.

In certain embodiments, the subject is undergoing secondary anti-hyperplastic therapy. Examples of such secondary anti-hyperplastic therapy include chemotherapy, radiotherapy, immunotherapy, phototherapy, cryotherapy, toxin therapy, hormonal therapy, or surgery. Any method of delivery of the composition is contemplated by the present invention. For example, the composition can be delivered systemically, intravascularly, subcutaneously, intradermally, or locally to a tumor mass.

Any antigen-presenting cell is contemplated by the present methods of preventing a disease. However, in certain embodiments, the antigen-presenting cell is a dendritic cell. The methods of the present invention can further involve culturing the antigen-presenting cells following electroporation of the mixture or measurement of the immune response of the antigen-presenting cell following electroporation. The immune response can be measured by any method known to one of skill in the art. For example, the immune response is monitored in vitro by ELISpot, ELISA, PCR, or tumor cell killing. Measurement of the immune response can also be performed in vivo by measurement of tumor size and immune monitoring pre- and post-treatment.

A still further object of the present invention is to provide for compositions including an antigen-presenting cell, wherein the antigen-presenting cell is loaded with one or more antigens using any of the methods that have been previously described in this summary and elsewhere in this specification. In a certain embodiment, the composition is a pharmaceutical composition suitable for delivery to a subject. The subject can be a human subject. Although compositions involving any antigen-presenting cell are contemplated by the present invention, in a certain embodiment the antigen-presenting cell is a dendritic cell.

Still further, the use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternative are mutually exclusive, although the disclosure supports a definition that refers to only alternatives and “and/or.”

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows FITC-Dextran results showing uptake of dextran into cells using electroporation.

FIG. 2 shows FITC-albumin results showing uptake of albumin into cells using electroporation.

FIG. 3 shows electroporation-mediated whole tumor cell lysate loaded DCs triggered a stronger T cell response than co-culturing. Human monocyte-derived DCs were either co-cultured or electroporated with tumor lysate. Then the DCs were co-cultured with autologous peripheral blood lymphocytes (PBLs) comprising T cells. T cells were re-stimulated with modified DCs 7 days later.

FIG. 4 shows whole tumor lysate loaded dendritic cells elicited auto T cell response. Whole tumor cell lysate DCs loaded by electroporation vs. lysate co-cultured Human monocyte-derived DCs were either co-cultured for 30 min (Co-CX 30 min) or overnight (Co-CX O/N), or loaded by electroporation with human melanoma cell line A-375 lysate at a DC vs. tumor cell ratio of 10:1. Loaded DCs were then washed with PBS (except the group of O/N) and incubated overnight with TNFα, IL-1 and PGE to induce maturation. The matured DCs were then incubated with autologous peripheral blood lymphocytes (a ratio of 1 DC: 10 PBL cells) in the presence of IL-2 and IL-7. PBLs were re-stimulated with additional modified DCs 10 days later. The conditioned tissue culture media was collected 18 hr post re-stimulation and analyzed for IFNγ production by commercially available ELISA kit (R&D System). Incubation of PBLs overnight with PHA (10 μg/mL) was positive control of IFN-γ production.

FIG. 5 shows electroporation-mediated whole tumor cell lysate loaded DCs prevented tumor challenge better than “pulsing” or co-culturing lysate with the DCs. BalbC mouse marrow CD34+ cells-derived DCs were either co-cultured (triangles) or electroporated (circles) with RENCA tumor lysate. Then the DCs were allowed to mature and were injected into syngeneic BalbC mice subcutaneously. Two weeks later, the mice were challenged by injection of RENCA tumor cells at a different site than that of the injection with DCs. Size of the tumors was measured starting 9 days post tumor challenge.

FIG. 6 shows induction of a primary, tumor specific CTL response in vitro using DC's electroporated with whole tumor lysate. Splenocytes from syngeneic C57B16 mice were stimulated with CD34+cell derived DCs electroporated or co-cultured with B16 melanoma cell lysate (1 B16: 10 DC), or electroporated without addition of lysate. The splenocytes were restimulated for another 3 rounds and then were incubated with ⁵¹Cr labeled B16 melanoma cells in a standard cytotoxicity assay.

FIG. 7 shows that whole tumor lysate loaded by electroporation DCs reduce Lewis lung metastases in a therapeutic model. Lewis lung carcinoma (LLC) cells were injected i.v. (tail vein) into C57BL6 mice. Isolated C57BL6 DCs were either co-cultured or electroporated with LLC whole tumor cell lysate and matured. As a control, DCs were electroporated in the absence of any lysate (no lysate). 3 days after LLC injection, DCs were injected by tail vein (8 mice/group). As a control, one group of mice was not given any DCs (no DC control). After an additional 3 days (day 6), a second dose of identically loaded DCs were injected into the same mice. On day 15 post-LLC injection, mice were sacrificed and lungs were dissected and weighed. The no tumor control group reflects normal lung weights of mice that were not challenged with any LLC. Administration of DCs that had been electroporated with LLC lysate caused a significant reduction in LLC lung metastases, as indicated by a significant decrease in lung weights.

### DETAILED DESCRIPTION OF THE INVENTION

The methods disclosed herein overcome the limitations of the prior art by providing improved techniques for immunotherapy of hyperproliferative disorders and other diseases caused by microorganisms, such as infectious diseases. It was discovered that electroporation of antigen-presenting cells (APCs) can be used to effectively load APCs with tumor antigens, including tumor lysates, as well as other microorganisms. Administration of these APCs to a subject with cancer can provide an effective form of immunotherapy against cancer. The invention presents an improvement over the prior art because, in one embodiment, there is no need to have a particular tumor associated-antigen (TAA) or pathogenic antigen identified since lysate contains essentially all the antigens that are present on the surface of the tumor cell or in its cytoplasm or in the microorganism or microorganism-infected cell. In addition, it is not necessary to identify the exact nature of the TAAs or pathogenic antigens or their concentrations in the lysate.

Use of whole tumor lysate to load APCs overcomes the requirement of defined epitopes. Tumor tissue, the source material for the preparation of cell lysate, may be limiting if treatment is initiated at an early stage of the disease and only a small tumor is present. Another advantage of loading APCs with lysate versus loading with a single antigen is the fact that there is no need to have T cell clones, since with single antigen loading, the T cells have to be stimulated to first recognize the antigen. The whole tumor lysate approach also avoids the “one antigen/epitope” problem, where many tumors have no well-established TAA available and, as a result, there is more likely to be facilitated class I and class II MHC presentation. Therefore, the invention provides novel forms of immunotherapy of cancer.

A. Treatment of Diseases

The invention may be used in the treatment and prevention of diseases including, but not limited to, infectious diseases and/or hyperproliferative diseases.

As used herein, the terms “treatment”, “treat”, “treated”, or “treating” refer to prophylaxis and/or therapy. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, e.g., reduce or eliminate the infection or prevent it from becoming worse. When used with respect to cancer, the treatment is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

1. Hyperproliferative Diseases

The invention may be used in the treatment and prevention of hyperproliferative diseases including, but not limited to, cancer. A hyperproliferative disease is any disease or condition which has, as part of its pathology, an abnormal increase in cell number. Included in such diseases are benign conditions such as benign prostatic hypertrophy and ovarian cysts. Also included are premalignant lesions, such as squamous hyperplasia. At the other end of the spectrum of hyperproliferative diseases are cancers. A hyperproliferative disease can involve cells of any cell type. The hyperproliferative disease may or may not be associated with an increase in size of individual cells compared to normal cells.

Another type of hyperproliferative disease is a hyperproliferative lesion, a lesion characterized by an abnormal increase in the number of cells. This increase in the number of cells may or may not be associated with an increase in size of the lesion. Examples of hyperproliferative lesions that are contemplated for treatment include benign tumors and premalignant lesions. Examples include, but are not limited to, squamous cell hyperplastic lesions, premalignant epithelial lesions, psoriatic lesions, cutaneous warts, periungual warts, anogenital warts, epidermdysplasia verruciformis, intraepithelial neoplastic lesions, focal epithelial hyperplasia, conjunctival papilloma, conjunctival carcinoma, or squamous carcinoma lesion. The lesion can involve cells of any cell type. Examples include keratinocytes, epithelial cells, skin cells, and mucosal cells. Cancer is one of the leading causes of death, being responsible for approximately 526,000 deaths in the United States each year. The term “cancer” as used herein is defined as a tissue of uncontrolled growth or proliferation of cells, such as a tumor.

Cancer develops through the accumulation of genetic alterations (Fearon and Vogelstein, 1990) and gains a growth advantage over normal surrounding cells. The genetic transformation of normal cells to neoplastic cells occurs through a series of progressive steps. Genetic progression models have been studied in some cancers, such as head and neck cancer (Califano et al., 1996). The present invention provides methods of treatment and prevention of cancer. Treatment and prevention of any type of cancer is contemplated by the present invention. The present invention also contemplates methods of prevention of cancer in a subject with a history of cancer. Examples of cancers have been outlined above.

2. Infectious Diseases

In certain embodiment of the invention, the present invention is useful for the treatment and/or prevention of infectious disease. Infectious diseases include infections of viral etiology such as HIV, influenza, Herpes, viral hepatitis, Epstein Bar, polio, viral encephalitis, measles, chicken pox, Papilloma virus etc.; or infections of bacterial etiology such as pneumonia, tuberculosis, syphilis, etc.; or infections of parasitic etiology such as malaria, trypanosomiasis, leishmaniasis, trichomoniasis, amoebiasis, etc.

B. Antigen Presenting Cells

In general, the term “antigen presenting cell” can be any cell that accomplishes the goal of the invention by aiding the enhancement of an immune response (i.e., from the T-cell or -B-cell arms of the immune system) against an antigen (e.g., a TAA). Examples of antigen-presenting cells include DCs, B-cells and macrophages. Such cells can be defined by those of skill in the art, using methods disclosed herein and in the art. In one embodiment of the invention, the APC is a DC.

DCs are the major APCs for initiation of immune responses. As DCs are unique in thier ability to activate naive [CD4+ AND CD8+ T] cells, they play a crucial role in priming both MHC class [II] and class I-restricted, antigen-specific T cell responses (Banchereau et al., 1998). However, exogenously introduced antigens, for example, those found in vaccines consisting of antigenic proteins or killed pathogens, are predominantly processed via the MHC class II pathway for presenting to [CD4+ T] cells (Moore et al., 1988). These types of vaccines stimulate potent humoral immunity but are relatively ineffective at stimulating [CD8+ CTL]. This shortcoming has led to an investigation of vaccine strategies that specifically target DCs to present antigens via MHC class I in addition to class II. DCs have been shown to possess a unique pathway for processing exogenous antigen, especially in particulate form, for presentation by the MHC class I pathway (Rodriguez et al., 1999).

As is understood by one of ordinary skill in the art, a cell that displays or presents an antigen normally or preferentially with a class II major histocompatability molecule or complex to an immune cell is an “antigen presenting cell.” In certain aspects, a cell (e.g., an APC cell) may be fused with another cell, such as a recombinant cell or a tumor cell that expresses the desired antigen. Methods for preparing a fusion of two or more cells is well known in the art, such as for example, the methods disclosed in Goding, 1986; Campbell, 1984; Kohler and Milstein, 1975; Kohler and Milstein, 1976, Gefter et al., 1977, each incorporated herein by reference. Fusion of antigen-presenting cells using electroporation has also been described (Scott-Taylor et al., 2000). In some cases, the immune cell to which an antigen presenting cell displays or presents an antigen to is a CD4+TH cell. Additional molecules expressed on the APC or other immune cells may aid or improve the enhancement of an immune response. Secreted or soluble molecules, such as for example, cytokines and adjuvants, may also aid or enhance the immune response against an antigen. Such molecules are well known to one of skill in the art, and various examples are described herein.

Any method of preparation of APCs known to one of skill in the art can be utilized in the present invention. Certain examples of techniques useful in the isolation, identification, preparation, and culturing of dendritic cells and other APCs are provided in U.S. Pat. Nos. 5,851,756, 5,994,126, 6,274,378, 6,051,432, 6,017,527, 6,080,409, 6,004,807 (each specifically incorporated by reference herein).

C. Antigens Useful in the Practice of the Present Invention

The term “antigen” as used herein is defined as a molecule that provokes an immune response. This immune response may involve either antibody production, the activation of specific immunologically-competent cells, or both. An antigen can be derived from organisms, killed or inactivated whole cells, or lysates. In general, antigens useful in the practice of the present invention include any antigen associated with a hyperproliferative cell, a microorganism (for example, viruses, bacteria, fungi, parasites), and/or any cell infected with a microorganism.

To this end, certain embodiments of the present invention comprise loading of APCs with a lysate, for example “cell lysate” and/or lysate from any microorganism (such as a virus). “Lysate” is defined herein to pertain to the material that results from application of a procedure to cause a disruption of the normal structure of the cell and/or microorganism. More particularly, “cell lysate” is defined herein to pertain to the cellular material that results from application of a procedure to cause a disruption of the normal cellular structure. Any method of preparation of a lysate is contemplated by the present invention. For example, preparation of a lysate by freeze-thaw techniques is one way in which a lysate can be prepared. One of skill in the art would be familiar with the wide range of techniques available in the preparation of lysates. Preparation of a lysate may or may not involve centrifugation with removal of a pellet prior to use in loading the APCs. A cell lysate can be a tumor cell lysate, wherein the cells are either benign tumor cells or cancerous (i.e., malignant) cells.

1. Hyperproliferative Cells

In the context of this disclosure and as used herein, an “antigen of a hyperproliferative cell” is defined as any protein or other substance having antigenic properties that are contained in the hyperproliferative cells. The antigen may or may not be a substantially isolated and purified antigen. As previously noted, the hyperproliferative cell can be a tumor cell, which can in turn be either a benign tumor cell or a malignant (i.e., cancer) cell.

The antigen of a hyperproliferative cell that is used in the present invention may, for example, be a tumor associated antigen. In the context of the present invention, a “tumor associated antigen” (TAA) is defined as any protein or other substance having antigenic properties that are contained in a tumor cell and are expressed differently than on normal cells. For example, TAAs can be membrane proteins or altered carbohydrate molecules of glycoproteins or glycolipids on the cell surface. The protein or other substance may be a substance that is normally expressed by the host cell that is mutated or has altered surface expression. Tumor cells expressing TAAs can be recognized by the body's immune system as though they were foreign cells. The body usually responds by mounting a cellular immune response to these antigens and the tumor cells on which they are displayed. “Tumor restricted antigens” (TRAs) include those antigens that are upregulated in tumor cells compared to normal cells or only expressed by tumor cells. Therefore, although any antigen of a hyperproliferative cell is contemplated by the present invention, preferred antigens would be TAAs or TRAs. However, as noted, any antigen that is contained in a hyperproliferative cell is contemplated by the present invention.

A number of purported TAAs have been identified. Examples include gp100, Melan-A/MART, MAGE-A, MAGE (melanoma antigen E), MAGE-3, MAGE-4, MAGEA3, tyrosinase, TRP2, NY-ESO-1, CEA (carcinoembryonic antigen), PSA, p53, Mammaglobin-A, Survivin, Mucl (mucin1)/DF3, metallopanstimulin-1 (MPS-1), Cytochrome P450 isoform 1B1, 90K/Mac-2 binding protein, Ep-CAM (MK-1), HSP-70, hTERT (TRT), LEA, LAGE-1/CAMEL, TAGE-1, GAGE, 5T4, gp70, SCP-1, c-myc, cyclin B1, MDM2, p62, Koc, IMP1, RCAS1, TA90, OA1, CT-7, HOM-MEL-40/SSX-2, SSX-1, SSX-4, HOM-TES-14/SCP-1, HOM-TES-85, HDAC5, MBD2, TRIP4, NY-CO-45, KNSL6, HIP1R, Seb4D, KIAA1416, IMP1, 90K/Mac-2 binding protein, MDM2, NY/ESO, and LMNA.

While the immune response that is mounted against the TAA may retard the growth of the cancer cells, it is often not able to fully arrest tumor growth because of poor accessibility of TAAs If this immune response can be made more vigorous or more specifically directed against TAAs, then cancer can be more effectively treated. To this end, certain embodiments of the present invention comprise loading of APCs with a cell lysate of a hyperproliferative cell.

2. Microorganisms

Antigens associated with a microorganism or a microorganism-infected cell can also be used in the present invention. As used herein, the term “microorganism” refers to a microscopic organism, for example bacteria, viruses, prions, fungi, parasites or protozoa. In certain embodiments, the microorganism is a “pathogenic microorganism” or “pathogen” in as much as the microorganism can cause disease when it infects a host, such as a human.

It is envisioned that lysates of microorganisms can be loaded into APCs. In certain embodiments, it may be advantageous to inactivate and/or attenuate the microorganism either prior to production of the lysate or after the production of the lysate of the microorganism. Microorganisms can be inactivated and/or attenuated via standard methods known and used in the art, for example, chemical treatments, i.e., formaldehyde and/or glutaraldehyde and/or heat. In addition to utilizing the lysate of the microorganism, lysates of cells infected with a microorganism can also be used in the present invention. The advantage of using lysates of microorganisms and/or microorganism-infected cells is that it eliminates or overcomes the problem of identifying and/or isolating a specific antigen or epitope.

The present invention would have applications therefore in the prevention and treatment of viral diseases by utilizing either the viruses themselves or virally-infected cells. The following pathogenic viruses which are mentioned by way of example, influenza A, B and C, parainfluenza, paramyxoviruses, Newcastle disease virus, respiratory syncytial virus, measles, mumps, adenoviruses, adenoassociated viruses, parvoviruses, Epstein-Barr virus (EBV), rhinoviruses, coxsackieviruses, echoviruses, reoviruses, rhabdoviruses, lymphocytic choriomeningitis, coronavirus, polioviruses, herpes simplex (HSV), human immunodeficiency viruses (HIV), cytomegaloviruses, papillomaviruses, human papillomavirus (HPV), hepatitis B virus (HBV), hepatitis C virus (HCV), varicella-zoster, poxviruses, rubella, rabies, picomaviruses, rotavirus and Kaposi associated herpes virus.

In addition to the viral diseases mentioned above, the present invention is also useful in the prevention, inhibition, or treatment of bacterial infections by utilizing either the bacteria or bacterially-infected cells. The following bacteria are mention by way of example, including, but not limited to, serotypes of pneumococci, streptococci such as S. pyogenes, S. agalactiae, S. equi, S. canis, S. bovis, S. equinus, S. anginosus, S. sanguis, S. salivarius, S. mitis, S. mutans, other viridans streptococci, peptostreptococci, other related species of streptococci, enterococci such as Enterococcus faecalis, Enterococcus faecium, Staphylococci, such as Staphylococcus epidermidis, Staphylococcus aureus, particularly in the nasopharynx, Hemophilus influenzae, pseudomonas species such as Pseudomonas aeruginosa, Pseudomonas pseudomallei, Pseudomonas mallei, brucellas such as Brucella melitensis, Brucella suis, Brucella abortus, Bordetella pertussis, Neisseria meningitidis, Neisseria gonorrhoeae, Moraxella catarrhalis, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, etc. Listeria monocytogenes, Nocordia asteroides, Bacteroides species, Actinomycetes species, Treponema pallidum, Leptospirosa species and related organisms. The invention may also be useful against gram negative bacteria such as Klebsiella pneumoniae, Escherichia coli, Proteus, Serratia species, Acinetobacter, Yersinia pestis, Francisella tularensis, Enterobacter species, Bacteriodes and Legionella species and the like.

Yet further, either fungal and other mycotic pathogens or cells infected with fungal or other mycotic pathogens may be used in the present invention to prevent and/or treat diseases, ranging from mycoses involving skin, hair, or mucous membranes, such as, but not limited to, Aspergillosis, Black piedra, Candidiasis, Chromomycosis, Cryptococcosis, Onychomycosis, or Otitis extema (otomycosis), Phaeohyphomycosis, Phycomycosis, Pityriasis versicolor, ringworm, Tinea barbae, Tinea capitis, Tinea corporis, Tinea cruris, Tinea favosa, Tinea imbricata, Tinea manuum, Tinea nigra (palmaris), Tinea pedis, Tinea unguium, Torulopsosis, Trichomycosis axillaris, White piedra. Fungal and mycotic pathogens that can be used in the present invention include, but are not limited to, Absidia spp., Actinomadura madurae, Actinomyces spp., Allescheria boydii, Alternaria spp., Anthopsis deltoidea, Apophysomyces elegans, Arnium leoporinum, Aspergillus spp., Aureobasidium pullulans, Basidiobolus ranarum, Bipolaris spp., Blastomyces dermatitidis, Candida spp., Cephalosporium spp., Chaetoconidium spp., Chaetomium spp., Cladosporium spp., Coccidioides immitis, Conidiobolus spp., Corynebacterium tenuis, Cryptococcus spp., Cunninghamella bertholletiae, Curvularia spp., Dactylaria spp., Epidermophyton spp., Epidermophyton floccosum, Exserophilum spp., Exophiala spp., Fonsecaea spp., Fusarium spp., Geotrichum spp., Helminthosporium spp., Histoplasma spp., Lecythophora spp., Madurella spp., Malassezia furfur, Microsporum spp., Mucor spp., Mycocentrospora acerina, Nocardia spp., Paracoccidioides brasiliensis, Penicillium spp., Phaeosclera dematioides, Phaeoannellomyces spp., Phialemonium obovatum, Phialophora spp., Phoma spp., Piedraia hortai, Pneumocystis carinii, Pythium insidiosum, Rhinocladiella aquaspersa, Rhizomucor pusillus, Rhizopus spp., Saksenaea vasiformis, Sarcinomyces phaeomuriformis, Sporothrix schenckii, Syncephalastrum racemosum, Taeniolella boppii, Torulopsosis spp., Trichophyton spp., Trichosporon spp., Ulocladium chartarum, Wangiella dermatitidis, Xylohypha spp., and Zygomyetes spp.

In addition, the invention may prove useful in controlling protozoan or macroscopic infections by organisms such as Cryptosporidium, Isospora belli, Toxoplasma gondii, Trichomonas vaginalis, Cyclospora species, for example, and for Chlamydia trachomatis and other Chlamydia infections such as Chlamydia psittaci, or Chlamydia pneumoniae, for example.

D. Preparation of Lysates

The lysates of the present invention can be prepared using any of the following standard techniques.

1. Detergents

Cells are bounded by membranes. In order to release components of the cell, it is necessary to break open the cells. The most advantageous way in which this can be accomplished, according to the present invention, is to solubilize the membranes with the use of detergents. Detergents are amphipathic molecules with an apolar end of aliphatic or aromatic nature and a polar end which may be charged or uncharged. Detergents are more hydrophilic than lipids and thus have greater water solubility than lipids. They allow for the dispersion of water insoluble compounds into aqueous media and are used to isolate and purify proteins in a native form.

Detergents can be denaturing or non-denaturing. The former can be anionic such as sodium dodecyl sulfate or cationic such as ethyl trimethyl ammonium bromide. These detergents totally disrupt membranes and denature the protein by breaking protein-protein interactions. Non-denaturing detergents can be divided into non-anionic detergents such as Triton®X-100, bile salts such as cholates and zwitterionic detergents such as CHAPS. Zwitterionics contain both cationic and anion groups in the same molecule, the positive electric charge is neutralized by the negative charge on the same or adjacent molecule.

Denaturing agents such as SDS bind to proteins as monomers and the reaction is equilibrium driven until saturated. Thus, the free concentration of monomers determines the necessary detergent concentration. SDS binding is cooperative i.e. the binding of one molecule of SDS increase the probability of another molecule binding to that protein, and alters proteins into rods whose length is proportional to their molecular weight.

Non-denaturing agents such as Triton®X-100 do not bind to native conformations nor do they have a cooperative binding mechanism. These detergents have rigid and bulky apolar moieties that do not penetrate into water soluble proteins. They bind to the hydrophobic parts of proteins. Triton®X100 and other polyoxyethylene nonanionic detergents are inefficient in breaking protein-protein interaction and can cause artifactual aggregations of protein. These detergents will, however, disrupt protein-lipid interactions but are much gentler and capable of maintaining the native form and functional capabilities of the proteins.

Detergent removal can be attempted in a number of ways. Dialysis works well with detergents that exist as monomers. Dialysis is somewhat ineffective with detergents that readily aggregate to form micelles because they micelles are too large to pass through dialysis. Ion exchange chromatography can be utilized to circumvent this problem. The disrupted protein solution is applied to an ion exchange chromatography column and the column is then washed with buffer minus detergent. The detergent will be removed as a result of the equilibration of the buffer with the detergent solution. Alternatively the protein solution may be passed through a density gradient. As the protein sediments through the gradients the detergent will come off due to the chemical potential.

Often a single detergent is not versatile enough for the solubilization and analysis of the milieu of proteins found in a cell. The proteins can be solubilized in one detergent and then placed in another suitable detergent for protein analysis. The protein detergent micelles formed in the first step should separate from pure detergent micelles. When these are added to an excess of the detergent for analysis, the protein is found in micelles with both detergents. Separation of the detergent-protein micelles can be accomplished with ion exchange or gel filtration chromatography, dialysis or buoyant density type separations.

a) Triton®X-Detergents:

This family of detergents (Triton®X-100, X114 and NP-40) have the same basic characteristics but are different in their specific hydrophobic-hydrophilic nature. All of these heterogeneous detergents have a branched 8-carbon chain attached to an aromatic ring. This portion of the molecule contributes most of the hydrophobic nature of the detergent. Triton®X detergents are used to solubilize membrane proteins under non-denaturing conditions. The choice of detergent to solubilize proteins will depend on the hydrophobic nature of the protein to be solubilized. Hydrophobic proteins require hydrophobic detergents to effectively solubilize them.

Triton®X-100 and NP-40 are very similar in structure and hydrophobicity and are interchangeable in most applications including cell lysis, delipidation protein dissociation and membrane protein and lipid solubilization. Generally 2 mg detergent is used to solubilize 1 mg membrane protein or 10 mg detergent/1 mg of lipid membrane. Triton®114 is useful for separating hydrophobic from hydrophilic proteins.

b) Brij® Detergents

These are similar in structure to Triton®X detergents in that they have varying lengths of polyoxyethylene chains attached to a hydrophobic chain. However, unlike Triton®X detergents, the Brij® detergents do not have an aromatic ring and the length of the carbon chains can vary. The Brij® detergents are difficult to remove from solution using dialysis but may be removed by detergent removing gels. Brij®58 is most similar to Triton®X100 in its hydrophobic/hydrophilic characteristics. Brij®-35 is a commonly used detergent in HPLC applications.

c) Dialyzable Nonionic Detergents

η-Octyl-β-D-glucoside (octylglucopyranoside) and η-Octyl-β-D-thioglucoside (octylthioglucopyranoside, OTG) are non-denaturing non-ionic detergents which are easily dialyzed from solution. These detergents are useful for solubilizing membrane proteins and have low UV absorbances at 280 nm. Octylglucoside has a high CMC of 23-25 mM and has been used at concentrations of 1.1-1.2% to solubilize membrane proteins.

Octylthioglucoside was first synthesized to offer an alternative to octylglucoside. Octylglucoside is expensive to manufacture and there are some inherent problems in biological systems because it can be hydrolyzed by β-glucosidase.

d) Tween® Detergents:

The Tween® detergents are non-denaturing, non-ionic detergents. They are polyoxyethylene sorbitan esters of fatty acids. Tween® 20 and Tween® 80 detergents are used as blocking agents in biochemical applications and are usually added to protein solutions to prevent nonspecific binding to hydrophobic materials such as plastics or nitrocellulose. They have been used as blocking agents in ELISA and blotting applications. Generally, these detergents are used at concentrations of 0.01-1.0% to prevent nonspecific binding to hydrophobic materials.

Tweeng® 20 and other nonionic detergents have been shown to remove some proteins from the surface of nitrocellulose. Tween® 80 has been used to solubilize membrane proteins, present nonspecific binding of protein to multiwell plastic tissue culture plates and to reduce nonspecific binding by serum proteins and biotinylated protein A to polystyrene plates in ELISA.

The difference between these detergents is the length of the fatty acid chain. Tween® 80 is derived from oleic acid with a C 18 chain while Tween® 20 is derived from lauric acid with a C 12 chain. The longer fatty acid chain makes the Tween® 80 detergent less hydrophilic than Tween® 20 detergent. Both detergents are very soluble in water.

The Tween® detergents are difficult to remove from solution by dialysis, but Tween® 20 can be removed by detergent removing gels. The polyoxyethylene chain found in these detergents makes them subject to oxidation (peroxide formation) as is true with the Triton® X and Brij® series detergents.

e) Zwitterionic Detergents

The zwitterionic detergent, CHAPS, is a sulfobetaine derivative of cholic acid. This zwitterionic detergent is useful for membrane protein solubilization when protein activity is important. This detergent is useful over a wide range of pH (pH 2-12) and is easily removed from solution by dialysis due to high CMCs (8-10 mM). This detergent has low absorbances at 280 nm making it useful when protein monitoring at this wavelength is necessary. CHAPS is compatible with the BCA Protein Assay and can be removed from solution by detergent removing gel. Proteins can be iodinated in the presence of CHAPS.

CHAPS has been successfully used to solubilize intrinsic membrane proteins and receptors and maintain the functional capability of the protein. When cytochrome P-450 is solubilized in either Triton® X-100 or sodium cholate aggregates are formed.

2. Non-Detergent Methods

In addition to the above detergent methods, various non-detergent methods may be employed to prepare the lysates of the present invention:

a) Freeze-Thaw

This has been a widely used technique for lysis cells in a gentle and effective manner. Cells are generally frozen rapidly in, for example, a dry ice/ethanol bath until completely frozen, then transferred to a 37° C. bath until completely thawed. This cycle is repeated a number of times to achieve complete cell lysis.

b) Sonication

High frequency ultrasonic oscillations have been found to be useful for cell disruption. The method by which ultrasonic waves break cells is not fully understood but it is known that high transient pressures are produced when suspensions are subjected to ultrasonic vibration. The main disadvantage with this technique is that considerable amounts of heat are generated. In order to minimize heat effects specifically designed glass vessels are used to hold the cell suspension. Such designs allow the suspension to circulate away from the ultrasonic probe to the outside of the vessel where it is cooled as the flask is suspended in ice.

c) High Pressure Extrusion

This is a frequently used method to disrupt microbial cell. The French pressure cell employs pressures of 10.4×10⁷ Pa (16,000 p.s.i) to break cells open. This apparatus consists of a stainless steel chamber which opens to the outside by means of a needle valve. The cell suspension is placed in the chamber with the needle valve in the closed position. After inverting the chamber, the valve is opened and the piston pushed in to force out any air in the chamber. With the valve in the closed position, the chamber is restored to its original position, placed on a solid based and the required pressure is exerted on the piston by a hydraulic press. When the pressure has been attained the needle valve is opened fractionally to slightly release the pressure and as the cells expand they burst. The valve is kept open while the pressure is maintained so that there is a trickle of ruptured cell which may be collected.

d) Solid Shear Methods

Mechanical shearing with abrasives may be achieved in Mickle shakers which oscillate suspension vigorously (300-3000 time/min) in the presence of glass beads of 500 nm diameter. This method may result in organelle damage. A more controlled method is to use a Hughes press where a piston forces most cells together with abrasives or deep frozen paste of cells through a 0.25 mm diameter slot in the pressure chamber. Pressures of up to 5.5×10⁷ Pa (8000 p. s.i.) may be used to lyse bacterial preparations.

e) Liquid Shear Methods

These methods employ blenders, which use high speed reciprocating or rotating blades, homogenizers which use an upward/downward motion of a plunger and ball and microfluidizers or impinging jets which use high velocity passage through small diameter tubes or high velocity impingement of two fluid streams. The blades of blenders are inclined at different angles to permit efficient mixing. Homogenizers are usually operated in short high speed bursts of a few seconds to minimize local heat. These techniques are not generally suitable for microbial cells but even very gentle liquid shear is usually adequate to disrupt animal cells.

f) Hypotonic/Hypertonic Methods

Cells are exposed to a solution with a much lower (hypotonic) or higher (hypertonic) solute concentration. The difference in solute concentration creates an osmotic pressure gradient. The resulting flow of water into the cell in a hypotonic environment causes the cells to swell and burst. The flow of water out of the cell in a hypertonic environment causes the cells to shrink and subsequently burst.

E. Electroporation Device

Certain embodiments of the present invention involve the use of electroporation to facilitate antigen entry into cells. As used herein, “electroporation” refers to application of an electrical current or electrical field to a cell to facilitate entry of an antigen or antigen composition into the cell.

Electroporation devices can be classified into at least two categories, static and flow formats. Static formats of electroporation devices encompass a specialized cuvette which contains molded-in electrodes in fluid contact with a fixed volume of target cells. The molecules of interest are placed between two electrodes and pulsed with high voltage.

One of skill in the art would understand that any method and technique of electroporation (i.e., static and/or flow) is contemplated by the present invention. However, in certain embodiments of the invention, electroporation may be carried out as described in U.S. Publication US20030073238A1 the entire disclosure of which is specifically incorporated herein by reference. In other embodiments of the invention, electroporation may be carried out as described in issued U.S. Pat. No. 5,612,207 (issued Mar. 18, 1997; specifically incorporated herein by reference), issued U.S. Pat. No. 5,720,921 (issued Feb. 24, 1998; specifically incorporated herein by reference), issued U.S. Pat. No. 6,074,605 (issued Jun. 13, 2000; specifically incorporated herein by reference); issued U.S. Pat. No. 6,090,617 (issued Jul. 18, 2000; specifically incorporated herein by reference); and issued U.S. Pat. No. 6,485,961 (issued Nov. 26, 2002; specifically incorporated herein by reference).

The present invention may use a flow electroporation apparatus for electrical treatment of suspensions of particles, especially including living cells, comprising a flow electroporation cell assembly having one or more inlet flow portals, one or more outlet flow portals, and one or more flow channels, the flow channels being comprised of two or more walls, with the flow channels further being configured to receive and transiently contain a continuous flow of particles in suspension from the inlet flow portals; and paired electrodes disposed in relation to the flow channels such that each electrode forms at least one wall of the flow channels, the electrodes further comprising placing the electrodes in electrical communication with a source of electrical energy, whereby suspensions of particles flowing through the channels may be subjected to an electrical field formed between the electrodes.

It is to be understood that the electroporation system used to practice the present invention can be used in conjunction with commercially available cell separation apparatus. These include, but are not limited to, Haemonetics Cell Saveo® 5 autologous blood recovery system, the Haemonetics OrthoPAT® System, the Haemonetics MCS®+ Apheresis System, the Cobe Spectra Apheresis System, the Trima® Automated Blood Component Collection System, the Gambro BCT System, and the Baxter Healthcare CS-3000 Plus blood cell separator.

F. Electroporation and Antigen Uptake

Electroporation allows for the introduction of non-permeable molecules into living cells (see Review by Mir, 2000). The molecules diffuse through the electropermeabilized areas of the cell membrane. DNA electroporation was originally described using simple generators that produce exponentially decaying pulses (Mir, 2000). Square-wave electric pulse generators were later developed that allowed, on the one hand, specification of the various electric parameters (pulse intensity, pulse length, number of pulses) (Rols and Teissié, 1990), and on the other hand, to obtain electroporation conditions under which a very large proportion of cells was simultaneously permealized and alive (Mir et al., 1988). Selection of parameters is dependent on the cell type being electroporated and physical characteristics of the molecules that are to be taken up by the cell. The examples below describe certain embodiments wherein particular electroporation settings are employed to facilitate uptake of cancer cell lysate by the APCs.

The present invention also contemplates methods of loading of one or more antigens into an antigen-presenting cell. Any APC is contemplated by the present invention. However, in certain embodiments, the APC is a dendritic cell. The assumed low concentration of TAA in whole tumor lysate requires that a considerable amount of tumor lysate be used per APC to be loaded in vitro by conventional co-incubation methods. Typically the ratio of whole-tumor lysate to DCs is 1:1 (i.e. one tumor cell in the tumor tissue used to make the lysate per dendritic cell to which the whole tumor lysate is added) (Chang et al., 2002). In some cases this ratio is 1:3, i.e., more whole tumor lysate must be prepared to load a desired number of DCs. The present invention, however, allows the use of less lysate per antigen presenting cell. Such methods may be particularly advantageous in situations where the amount of antigenic material is limiting such as, for example, during the early stages of disease or infection. In regard to the treatment of cancer, 1 gram of tumor is typically required for conventional co-culture methods for loading APCs. For many types of cancer 1 gram of tumor is not present or is not readily accessible. The present invention provides methods for loading APCs using less than 1 gram of tumor. For example, in certain embodiments of the invention APCs may be loaded using 1/10 gram or less of a tumor or even 1/100 gram or less of a tumor.

G. Cellular Vaccines

In certain embodiments of the invention, an APC loaded with one or more antigens may comprise the vaccine. The APCs may be isolated from a culture, tissue, organ or organism and administered to a subject as a cellular vaccine. Thus, the present invention contemplates a “cellular vaccine.” As used herein, the term “vaccine” refers to a formulation which contains the composition (loaded APCs) of the present invention and which is in a form that is capable of being administered to a subject. Typically, the vaccine comprises a conventional saline or buffered aqueous solution medium in which the composition of the present invention is suspended or dissolved. In this form, the composition of the present invention can be used conveniently to prevent, ameliorate, or otherwise treat a condition. Upon introduction into the subject or host, the vaccine is able to provoke an immune response including, but not limited to, the production of antibodies, cytokines and/or other cellular responses. One of skill in the art is also aware that the vaccine of the present invention may comprise all or part of the cell.

In certain embodiments, the APCs may be isolated from the subject that is to be vaccinated. Techniques that are well-known to those of skill in the art may be used to isolate the APCs. For example, the APCs can then be electroporated with a cancer cell lysate, matured ex vivo, and then cultured. Thereafter, the APCs can then be administered to the subject as a cellular vaccine.

Optionally, the vaccine of the present invention additionally includes an adjuvant which can be present in either a minor or major proportion relative to the compound of the present invention. The term “adjuvant” as used herein refers to non-specific stimulators of the immune response or substances that allow generation of a depot in the host which when combined with the vaccine of the present invention provide for an even more enhanced immune response. A variety of adjuvants can be used. Examples include incomplete Freund's adjuvant, aluminum hydroxide and modified muramyldipeptide. The term “adjuvant” as used herein also refers to typically specific stimulators of the immune response which when combined with the vaccine of the present invention provide for an even more enhanced and typically specific immune response. Examples include, but limited to, GM-CSF, IL-2, IL-12, IFNα. Further examples are within the knowledge of the person skilled in the art.

There is currently a need for improved vaccines that stimulate T cell-, and particularly cytotoxic T lymphocyte (CTL)-, mediated immunity against cell-associated or endogenous antigens. Targets for these vaccines may include microorganism-infected cells (i.e., cells infected with viruses, intracellular bacteria and parasites), as well as cancers. The initiation of CTL-mediated immunity requires that antigenic peptides be presented in association with major histocompatibility (MHC) class I molecules on the surface of APCs and, in particular, DCs (Ridge et al., 1998). Co-culturing of DCs with antigen mediates phagocytosis of the antigen resulting in MHC class II presentation. Electroporation can transport the antigen directly into the cytoplasm of DCs, giving rise to class I presentation. Thus, the present invention provides an improved vaccine that stimulates T cell-mediated immunity.

H. Immunodetection Methods

In certain embodiments, the present invention concerns immunodetection methods for measurement of the immune response of APCs. One of ordinary skill in the art would be familiar with a wide variety of immunodetection techniques that are available. Examples of immunodetection methods include enzyme linked immunosorbent assay (ELISA), ELISpot, radioimmunoassay (RIA), immunoradiometric assay, fluoroimmunoassay, chemiluminescent assay, bioluminescent assay, and Western blot, to mention a few. The steps of various useful immunodetection methods have been described in the scientific literature, such as, e.g., Doolittle and Ben-Zeev, 1999; Gulbis and Galand, 1993; De Jager et al., 1993; and Nakamura et al., 1987, each incorporated herein by reference.

In terms of antigen detection, the biological sample analyzed may be any sample that is suspected of containing an antigen, such as, for example, a dendritic cell, a homogenized tissue extract, or even any biological fluid that comes into contact with the cell, including blood and/or serum.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

I. Cell Culture

In certain embodiments of the invention, cell culture may be utilized in preparation of the APCs. In eukaryotic cell culture systems, the culture of the cells is generally under conditions of controlled pH, temperature, humidity, osmolarity, ion concentrations, and exchange of gases. Regarding the latter, oxygen and carbon dioxide (carbon dioxide) are of particular importance to the culturing of cells. In a typical eukaryotic cell culture system, an incubator is provided in which carbon dioxide is infused to maintain an atmosphere of about 5% carbon dioxide within the incubator. The carbon dioxide interacts with the tissue culture medium, particularly its buffering system, in maintaining the pH near physiologic levels. Conventional cell culture containers comprise tissue culture flasks, tissue culture bottles, and tissue culture plates. Additionally, for the culture of DCs, sterile Teflon-coated bags have been used to prevent cell attachment. Entry of carbon dioxide from the incubator atmosphere into a tissue culture plate generally involves a loosely fitting cover which overhangs the plate in excluding particulate contaminants from entering the plate chamber(s), but allows gas exchange between the incubator atmosphere and the atmosphere within the tissue culture plates. Similarly, for a tissue culture flasks or bottle, a loosely fitting cap excludes particulate contaminants from entering the chamber of the flask or bottle, but allows gas exchange between the incubator atmosphere and the atmosphere within the flask or bottle. More recently, a cap is provided with a gas permeable membrane or filter, thereby allowing for gas exchange with a tightly fitting cap.

In addition to carbon dioxide, the culturing of cells is dependent upon the ability to supply to the cells a sufficient amount of oxygen necessary for cell respiration and metabolic function. The supply of oxygen for cell respiration in conventional cell culture containers is in the header space of the container, e.g., the void space in the container that is above the surface of the tissue culture medium. Efforts to increase oxygen concentration to the cultured cells includes mechanical stirring, medium perfusion or aeration, increasing the partial pressure of oxygen, and/or increasing the atmospheric pressure. Thus, in conventional cell culture containers the volume or surface provided for gas exchange, as relative to the volume or surfaces of the whole container, is either inefficiently used and/or results in limiting the rate of gas exchange or in the equilibration of gases. This is even more noticeable in small-scale cultures (15 ml or less) in which rate of cell growth, cell densities, and total cell numbers, are frequently low due to space, surface area, and gas exchange limitations.

Any method of culturing APCs known to one of skill in the art can be utilized in the present invention. Certain examples of techniques used for culturing dendritic cells are provided in U.S. Pat. Nos. 5,851,756, 5,994,126, 6,274,378, 6,051,432, 6,017,527, 6,080,409, 6,004,807 (each specifically incorporated by reference herein).

J. Pharmaceutical Preparations

1. Formulations

Pharmaceutical preparations of APCs loaded with cancer cell antigens or antigens of microorganisms or antigens of microorganism-infected cells for administration to a subject are contemplated by the present invention. One of ordinary skill in the art would be familiar with techniques for administering cells such as APCs to a subject. As previously noted, the APCs may be cells that were grown in cell culture. One of ordinary skill in the art would be familiar with techniques are pharmaceutical reagents necessary for preparation of these cell prior to administration to a subject.

In certain embodiments of the present invention, the pharmaceutical preparation will be an aqueous composition that includes the APCs that have been loaded with a lysate, for example cancer cell lysate, microorganism lysate or microorganism-infected cell lysate. In certain other embodiments, the lysate is prepared using cells (i.e., cancer cells) that have been obtained from the subject. However, cells obtained from any source are contemplated by the present invention. In certain embodiments, cancer cells may have been obtained as a result of previous cancer surgery performed on the subject as part of the overall cancer treatment protocol that is specific for the particular patient.

Aqueous compositions of the present invention comprise an effective amount of a solution of the APCs in a pharmaceutically acceptable carrier or aqueous medium. As used herein, “pharmaceutical preparation” or “pharmaceutical composition” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the APCs, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The biological material should be extensively dialyzed to remove undesired small molecular weight molecules and/or lyophilized for more ready formulation into a desired vehicle, where appropriate. The APCs will then generally be formulated for administration by any known route, such as parenteral administration. Determination of the number of cells to be administered will be made by one of skill in the art, and will in part be dependent on the extent and severity of cancer, and whether the APCs are being administered for treatment of existing cancer or prevention of cancer and/or treatment or prevention of a disease caused by a microorganism. The preparation of the pharmaceutical composition containing the APCs of the invention disclosed herein will be known to those of skill in the art in light of the present disclosure.

An agent or substance of the present invention can be formulated into a composition at an appropriate pH. A person of ordinary skill in the art would be familiar with techniques for preparations for administration of the APCs, including techniques pertaining to preparation of APCs in a solution at an appropriate pH and with appropriate reagents to maintain cellular viability.

The present invention contemplates APCs loaded with lysate (i.e., tumor lysate) that will be in pharmaceutical preparations that are sterile solutions for subcutaneous injection, intramuscular injection, intravascular injection, intratumoral injection, or application by any other route. A person of ordinary skill in the art would be familiar with techniques for generating sterile solutions for injection or application by any other route.

Upon formulation, solutions will be administered in a manner compatible with the dosage formulation and in such amount as is therapeutically effective. The formulations are easily administered in a variety of dosage forms, such as the type of injectable solutions described above. For parenteral administration, the solution including the APCs should be suitably buffered. In this connection, sterile aqueous media which can be employed will be known to those of skill in the art in light of the present disclosure. The active agents disclosed herein may be formulated within a therapeutic mixture to comprise an appropriate number of APCs to be determined by one of ordinary skill in the art. The APCs may be administered with other agents that are part of the therapeutic regiment of the subject, such as other immunotherapy or chemotherapy.

2. Dosage

The present invention contemplates administration of APCs loaded with lysate (i.e., cancer cell lysate, microorganism lysate, or microorganism-infected cell lysate) to a subject for the treatment and prevention of cancer or any disease caused by a microorganism, such as an infectious disease. In certain embodiments, an effective amount of the APCs loaded with cancer cell lysate is determined based on the intended goal, for example tumor regression. For example, where existing cancer is being treated, the number of cells to be administered may be greater than where administration of APCs is for prevention of cancer. One of ordinary skill in the art would be able to determine the number of cells to be administered and the frequency of administration in view of this disclosure. The quantity to be administered, both according to number of treatments and dose, also depends on the subject to be treated, the state of the subject and the protection desired. Precise amounts of the therapeutic composition also depend on the judgment of the practitioner and are peculiar to each individual. Frequency of administration could range from 1-2 days, to 2-6 hours, to 6-10 hours, to 1-2 weeks or longer depending on the judgment of the practitioner.

Longer intervals between administration and lower numbers of cells may be employed where the goal is prevention. For instance, numbers of cells administered per dose may be 50% of the dose administered in treatment of active disease, and administration may be at weekly intervals. One of ordinary skill in the art, in light of this disclosure, would be able to determine an effective number of cells and frequency of administration. This determination would, in part, be dependent on the particular clinical circumstances that are present (e.g., type of disease (i.e., cancer, infectious disease, etc.), severity of the disease (i.e., cancer, infection, etc.).

In certain embodiments, it may be desirable to provide a continuous supply of the therapeutic APC compositions to the patient. Continuous perfision of the region of interest (such as the tumor, or infection site) may be preferred. The time period for perfusion would be selected by the clinician for the particular patient and situation, but times could range from about 1-2 hours, to 2-6 hours, to about 6-10 hours, to about 10-24 hours, to about 1-2 days to about 1-2 weeks or longer. Generally, the dose of the therapeutic composition via continuous perfusion will be equivalent to that given by single or multiple injections, adjusted for the period of time over which the doses are administered.

K. Combination Treatments

In order to increase the effectiveness of the APCs loaded (also referred to herein as “loaded APCs”) with cancer cell antigens or antigens of microorganisms or antigens of microorganism-infected cells, it may be desirable to combine the treatment using these loaded APCs with other agents effective in the treatment of cancer and/or infectious diseases.

1. Cancer

An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the loaded APCs and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the loaded APCs and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with immunotherapy. In the context of the present invention, it is contemplated that APC therapy could be used similarly in conjunction with chemotherapeutic, radiotherapeutic, or other immunotherapeutic intervention.

Alternatively, the immunotherapy with APCs may precede or follow the other agent treatment by intervals ranging from minutes to weeks. In embodiments where the other agent and loaded APCs are applied separately to the tumor cell or subject, one would generally ensure that a significant period of time did not expire between the time of each delivery, such that the agent and loaded APCs would still be able to exert an advantageously combined effect on the tumor cell. In such instances, it is contemplated that one may contact the tumor cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, APC therapy is “A” and the secondary agent, such as radio- or chemotherapy, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the loaded APCs of the present invention to a patient will follow general protocols for the administration of chemotherapeutics, taking into account the toxicity, if any, of the cells. It is expected that the treatment cycles would be repeated as necessary. It also is contemplated that various standard therapies, as well as surgical intervention, may be applied in combination with the described hyperproliferative cell therapy.

a). Chemotherapy

Cancer therapies also include a variety of combination therapies with both chemical and radiation based treatments. One of ordinary skill in the art would be familiar with the range of chemotherapeutic agents and combinations that are available. Chemotherapeutic agents include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein tansferase inhibitors, transplatinum, 5-fluorouracil, vincristin, vinblastin and methotrexate, or any analog or derivative variant of the foregoing.

b). Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

c). Immunotherapy

The APCs of the present invention may be administered in combination with other forms of immunotherapy. Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

d). Genes

The secondary treatment may be a gene therapy. For example, the gene therapy can be a vector encoding either a full length or truncated tumor cell antigen.

e). Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. As previously noted, resected tumor can be used in the generation of cancer cell lysate that is used to load the APCs used in the treatment of the cancer patient. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

f). Other Agents

It is contemplated that other agents may be used in combination with the present invention to improve the therapeutic efficacy of treatment. These additional agents include other immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-lalpha, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion, such as integrin and cadherin blocking antibodies, are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

2. Anti-Microbial Agents

In certain embodiments, an “antimicrobial agent” can be used in combination with the APCs loaded with a microorganism to enhance the effectiveness of the vaccine. An antimicrobial agent may comprise an antibiotic, anti-fungal, and anti-viral agent.

Antibiotics inhibits the growth of microorganisms without damage to the host. For example, the antibiotic may inhibit cell wall synthesis, protein synthesis, nucleic acid synthesis, or alter cell membrane function. Classes of antibiotics that can possibly be used in conjunction with the APCs include, but are not limited to, macrolides (i.e., erythromycin), penicillins (i.e., nafcillin), cephalosporins (i.e., cefazolin), carbepenems (i.e., imipenem, aztreonam), other beta-lactam antibiotics, beta-lactam inhibitors (i.e., sulbactam), oxalines (i.e. linezolid), aminoglycosides (i.e., gentamicin), chloramphenicol, sufonamides (i.e., sulfamethoxazole), glycopeptides (i.e., vancomycin), quinolones (i.e., ciprofloxacin), tetracyclines (i.e., minocycline), fusidic acid, trimethoprim, metronidazole, clindamycin, mupirocin, rifamycins (i.e., rifampin), streptogramins (i.e., quinupristin and dalfopristin) lipoprotein (i.e., daptomycin), polyenes (i.e., amphotericin B), azoles (i.e., fluconazole), and echinocandins (i.e., caspofungin acetate).

Anti-viral agents can also be used in combination with the loaded APCs to treat and/or prevent a viral infection or disease. Such anti-viral agents include, but are not limited to protease inhibitors (e.g., saquinavir, ritonavir, amprenavir), reverse transcriptase inhibitors (e.g., azidothymidine (AZT), lamioridine (3TC), dideoxyinosine (ddl)), dideoxycytidine (ddC), zidovudine), nucleoside analogs (e.g., acyclovir, penciclovir).

In certain embodiments, anti-fungul agents can be used in combination with the loaded APCs to treat and/or prevent a fungal infection. Such anti-fungal agents include, amphotericin B (Amphocin®, Fungizone®), butoconazole (Femstat®), clotrimazole (Mycelex®, Gyne-Lotrimin®, Lotrimin®, Lotrisone®), fluconazole (Diflucan®), flucytosine (Ancobon®), griseofulvin (Fulvicin P/G®, Grifulvin V®, Gris-PEG®), itraconazole (Sporanox®), ketoconazole (Nizoral®, miconazole (Femizol-M®, Monistat®), nystatin (Mycostatin®), terbinafine (Lamisil®), terconazole (Terazol®), or tioconazole (Vagistat®).

### L. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Isolation of Murine DCs

Mouse bone-marrow derived DCs were isolated from 8-12 day old Balb/C mice. Tibia and femur of the mice were removed and cleaned. The bone marrow cells were collected by flushing the bones with tissue culture media. The cells were pelleted by centrifugation and resuspended in red blood cell lysis buffer (ACK, Sigma). Cells were washed twice with PBS and then plated a 5×10⁶ cells/ml in AIM-V media supplemented with L-glutamine, penicillin and streptomycin, human serum albumin at 0.2%, mouse GM-CSF at 30 ng/ml, and mouse IL-4 at 10 ng/ml. After three days of culture GM-CSF and IL-4 were added to the cultures to final concentrations of 25 ng/ml and 10 ng/ml, respectively. After an additional 3 days of culture (day 6) the media was replaced with fresh media containing GM-CSF and IL-4 at 25 ng/ml and 10 ng/ml, respectively, together with the other supplements previously used. After an additional 2-4 days the cells in suspension and all adherent cells, the latter collected by trypsinization, were pooled and counted. The pooled cells were analyzed for the presence of the following markers by fluorescence activated cell sorting (FACS): CD3, CD14, MHC Class II markers, CD80, CD86, and CD11c. Based on the percentages of cells expressing these markers approximately 85% of the pooled cells were DCs. (The cells can be frozen for later use by standard cryogenic freezing techniques).

### Example 2

### Isolation of Human DCs

Human monocyte-derived DCs were isolated from human peripheral blood by centrifugation using standard procedures. The isolated macrophages (approximately 5×10⁸) were washed and plated at 5×10⁶ per milliliter in AIM-V media (Invitrogen) supplemented with L-glutamine, penicillin and streptomycin at standard concentrations used for tissue culture, human serum albumin at 0.2%, 2.0% autologous plasma, 30 ng/ml human GM-CSF, and 10 ng/ml human IL-4 (the latter two growth factors from R & D Systems). The average number of cells per surface area was 10⁸/185 cm².

After three days of culture GM-CSF and IL-4 were added to the cultures to final concentrations of 25 ng/ml and 10 ng/ml, respectively. After an additional 3 days of culture (day 6) the media was replaced with fresh media containing GM-CSF and IL-4 at 25 ng/ml and 10 ng/ml, respectively together, with the other supplements. After an additional 2-4 days, the cells in suspension and all adherent cells, the latter collected by trypsinization, were pooled and counted. The pooled cells were analyzed for the presence of the following markers by Fluorescence Activated Cell Sorting (FACS): CD3, CD14, MHC Class II markers, CD80, CD86, and CD1a. Based on the percentages of cells expressing these markers approximately 85% of the pooled cells were DCs. Cells were frozen at this point by cryogenic storage. For use, cells were thawed quickly using a 37° C. water bath and collected in AIM-V media. Cells were spun 10 min at 1000×g and counted and brought to 5×10⁷ cells/ml in EP buffer (EP buffer:125 mM KCl, 15 mM NaCl, 25 mM HEPES, 1.2 mM MgCl₂, 3 mM Glucose). The pooled DCs to be loaded with whole cell lysate by electroporation or for use as a control sample that would be electroporated but would not be loaded with whole cell lysate, and were resuspended at a concentration of 5×10⁷ cells/ml in EP buffer.

### Example 3

### Preparation of Lysate from RENCA, B16-F10, LLC or A375 Tumor Cells

The mouse renal carcinoma cell line (RENCA), melanoma (B16-F10), Lewis lung carcinoma (LLC) or A375 human melanoma cells were cultured in vitro, grown and collected by trypsinization, washed in phosphate buffered saline (PBS), and then 100×10⁶ cells were resuspended in a 1 ml final volume giving 100×10⁶ cells/ml. The cells were then lysed by freeze/thawing by subjecting them to 5 cycles of rapid freezing and thawing using a dry-ice/alcohol bath and a 37° C. water bath. Tumor lysate was also prepared by injecting mice with 1×10⁶ of these tumor cells, waiting 1-2 weeks to allow for the tumor to grow subcutaneously, and then the resulting tumor mass was dissected and subjected to the same freeze/thawing cycles as described above. After freeze-thawing, the lysates were centrifuged for 10 min at 13,000×g at room temperature, and the supernatants were transferred to 1.5 ml plastic centrifuge tubes (Eppendorf). The supernatants were remove and frozen at negative 80° C. for later use. From the starting number of cells and the final volume of lysate recovered, a concentration of cell equivalent material per milliliter was calculated. This calculation was not adjusted to account for elimination of cell material pelleted with the centrifugation that follows the freeze/thaw process.

### Example 4

### Preparation of DCs for Antigen Loading

The pooled DCs to be loaded with cell lysate by electroporation or for use as a control sample that would be electroporated but would not be loaded with cell lysate, were resuspended at a concentration of 5×10⁷ cells/ml in EP buffer, at a total volume of 200 μl. Tumor cell lysate was added to a mix with the above DC's suspended in EP buffer to provide one cell equivalent of tumor cell lysate per 10 DCs.

### Example 5

### Method of Cell Loading of Human DCs using A375 Human Melanoma Lysate

To cells in EP buffer, whole tumor lysate prepared from A375 human melanoma cells according to the method described above was added at a ratio of 10 DCs per cell equivalent of tumor cell lysate, and in the latter case a ratio of one DC per cell equivalent of tumor cell lysate. The DCs and tumor cell lysate were electroporated in a cuvette having gold coated electrodes spaced 3 mm apart by four pulses at intervals of 1 sec, each pulse having a voltage of 600 volts and a duration of 400 microseconds. Following electroporation, the cells were incubated for 20 minutes at 37° C. A suspension of DCs to which no tumor cell lysate was added were electroporated under the above conditions. Electroporated DCs were plated overnight (24hr) with 5 ng/ml TNFα, 1 μg/ml PGE and 1 ng/ml IL-1β to mature the DCs. The next day, the DCs were collected and counted again, and washed once with AIM-V media.

Peripheral blood lymphocytes (PBLs) from the same donor from whom DCs were isolated were isolated and cultured in AIM-V media supplemented with 2% autologous plasma. T cells were collected and counted. The above PBLs were resuspended in AIM-V containing 20 U/ml IL-2 and 10 ng/ml IL-7 at a concentration of 5×10⁶ per milliliter. To wells of a standard 96 well microtiter plate, 100 microliters of DCs that were electroporated or co-cultured with whole tumor lysate or electroporated without the addition of whole tumor lysate were added, 5×10⁴ cells per well. To each well containing DCs, 100 μl of a suspension of PBLs prepared as above were added, 5×10⁵ cells per well. To additional wells only PBLs were added, only DCs were added, or PBLs stimulated with 10 μg/ml PHA (phytohemagglutinin) were prepared to serve as controls.

DCs that were not used in the above procedure were frozen cryogenically. The above mixtures of DCs and PBLs (or in the case of controls the single type of cell added to the well) were incubated for seven days under standard cell culture conditions. After seven days, the above frozen DCs were thawed and recounted. These cells were transferred to AIM-V media containing IL-2 and IL-7 at 5×10⁵ cells/ml. One hundred microliters of these thawed DCs were added to each well that had previously received DCs. The cells were incubated an additional two days. The cells were then centrifuged at 2400×g for 5 minutes and the supernatant was collected. The pellet was resuspended in 200 μl of AIM containing 10 ng/ml IL-2.

### Example 6

### Measurement of Electroporated Cells Following Antigen Loading Using In vitro ELISPOT Assay

The resuspended cells were transferred into an ELISPOT plate containing a filter coated with anti-IFNgamma antibody according to the manufacturer's instructions and incubated overnight at 37° C. The remaining steps of the ELISPOT assay were performed per standard procedure per the manufacturer's instructions and spots resulting from the assay were detected and enumerated using a dissecting microscope.

Results from the above show that stimulation of DCs by electroporation in the presence of tumor lysate resulted in higher stimulation of T cells, as evidenced by the ELISPOT and ELISA assays, than did co-incubation of DCs with tumor lysate absent electroporation.

### Example 7

### FITC-Albumin and FITC-Dextran Loading of DCs

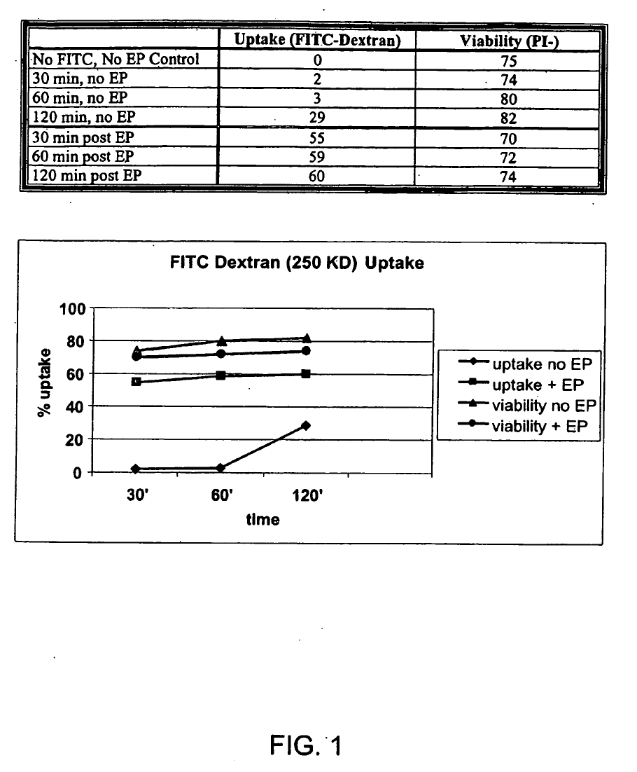

DCs can be loaded with fluorescein isothiocyanate (FITC) conjugated Albumin (MW approx. 68 kD) and FITC labeled Dextran (MW 250 kD), and co-culturing can be compared to electroporation for various periods of incubation. DCs (as confirmed by CD1a and class II MHC expression) were incubated with 1 mg/ml FITC-Dextran at a cell concentration of approximately 2×10⁷ cells/ml in electroporation buffer. Cells were either incubated at 37° C. or electroporated (15 μl/EP). Following EP, cells were allowed to recover at 37° C. After various lengths of time (30 min, 1 hr, 2 hr), cells were washed 3× with warmed PBS, followed by incubation in complete media. After the last timepoint, cells were collected and analyzed by flow cytometry for FITC-Dextran uptake and cell viability. Cell viability was unaffected by EP for all timepoints evaluated (FIG. 1). No uptake was observed in the absence of EP (‘no EP’), whereas 55-60% uptake was observed for EP (‘post EP’).

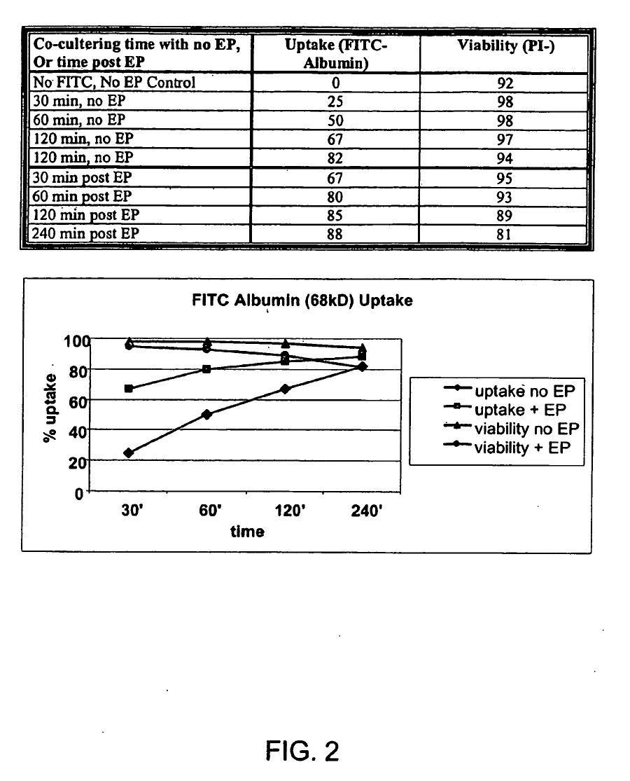

The experiment using FITC-Albumin was similar to the experiment using FITC-Dextran, except 0.5 mg/ml FITC-Albumin was used and the 4-hour time point was included. Cell viability was not significantly affected by the electroporation (FIG. 2). Uptake of FITC-Albumin plateaued by 1 hr at 80% for electroporated cells (FIG. 2). Co-cultured cells had comparable uptake by 4 hours.

### Example 8

### Tumor Cell Lysate Loaded Human DCs Elicited T Cell Response

Human monocyte-derived DCs were isolated as described above. The DCs were treated with cytokines (human GMCSF, human IL-4) for 7 days, and FACS was performed for DC markers (MHC, CD1a, CD80/CD86) as well as lack of expression of other markers (CD3, CD14). Tumor lysate of A375 melanoma was prepared using the technique described above, and stored frozen until use. DCs were then co-cultured with tumor lysate in EP buffer, using DC:tumor cell equivalents of 10:1 and 1:1. Cells were then either electroporated or simply incubated at 37° C. for co-incubating.

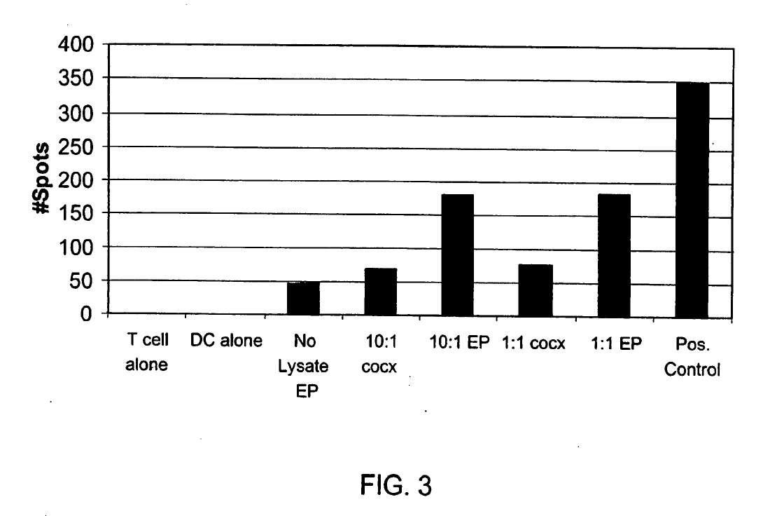

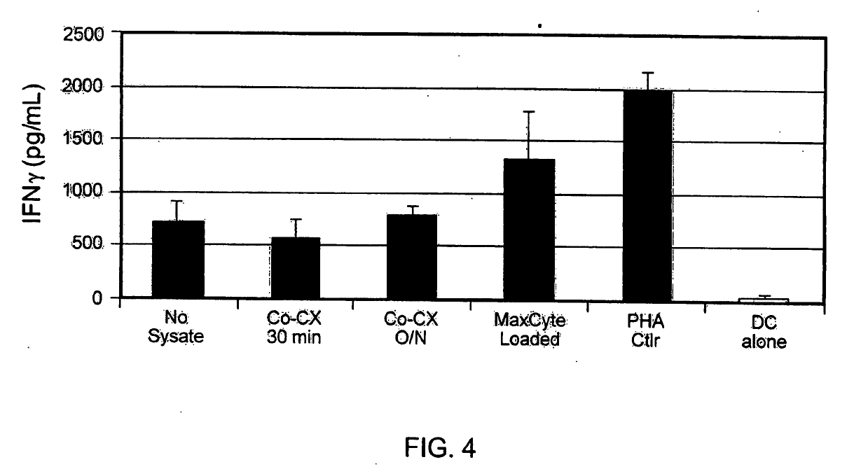

After 30 min, all cells were centrifuged and washed with PBS. DCs were then plated with media containing TNFα, IL-1, and PGE to mature the DCs. After 24 hours of incubation, the DCs and autologous PBL including T cells were collected and co-cultured in 96 wells at various ratios with IL-2 and IL-7. A portion of the DCs were frozen for later re-stimulation. The ratio of DC to T cells used was 1:100. After 1 week, the T cells were re-stimulated by adding the antigen-loaded DCs that had been previously frozen in the presence of IL-2. After a total of 2 weeks, supernatant was collected for IFNy production by ELISA and the T cells were transferred to an ELISPOT assay plate. DCs do not produce any IFNγ. Experimental controls included T cells only, T cells stimulated with phorbol ester as a positive control, and DCs only. Other controls include co-incubating T cells with DCs that have not been mixed with any tumor lysate. Results in FIG. 3 show that electroporation-mediated whole tumor cell lysate loaded DCs triggered a stronger T cell response than co-cultivation. FIG. 4 demonstrates that whole tumor lysate loaded DCs elicited a stronger auto T cell response than with co-cultivation.

### Example 9

### Immunotherapy of Mice Using Antigen-Loaded Murine DCs

Isolation of mouse bone marrow DCs was performed as previously described. The isolated DCs were loaded with murine renal carcinoma (RENCA) tumor lysate as previously described. Following electroporation the cells were incubated for 20 minutes at 37° C. In parallel a mixture of DCs and tumor lysate was prepared as for electroporation but was co-cultured for 30 minutes at 37° C. instead of being electroporated. The DCs of each mixture were transferred to 2 ml of AIM-V media supplemented with mouse GM-CSF at 50 ng/ml, mouse TNF alpha at 50 ng/ml, PGE (1 μg/ml) and hIL-1β (1 ng/ml)-(human IL-1 cross reacts with mouse) plated, and incubated at 37° C. overnight. The next day, the above DCs were collected by trypsinization, counted, washed once in PBS, and resuspended in PBS to a concentration of 1×10⁷ cells/ml. One hundred microliters of this cell suspension of the above cells (1×10⁶ cells) were injected subcutaneously into the left side of the backs of Balb/C mice.

A total of 20 mice were injected, with 5 mice receiving no DCs at all, 5 mice receiving DCs that had been electroporated in the absence of tumor cell lysate, 5 mice receiving DCs that had been co-cultured, but which had not been electroporated with tumor cell lysate, and 5 mice receiving DCs that had been electroporated in the presence of tumor cell lysate. After 12 days, all 20 mice were injected with 1×10⁵ RENCA cells grown in culture into the right side of the back (total volume per injection of 100 microliters). Beginning 10 days after injection of RENCA cells, tumors were measured bi-weekly using mechanical calipers by measuring the perpendicular axes of tumors (giving area or mm²) at or near the site of RENCA cell injection. Tumor volumes of each tumor were calculated with following standard formula: volume=π×length×width²/6 (Heller et al., 2002).

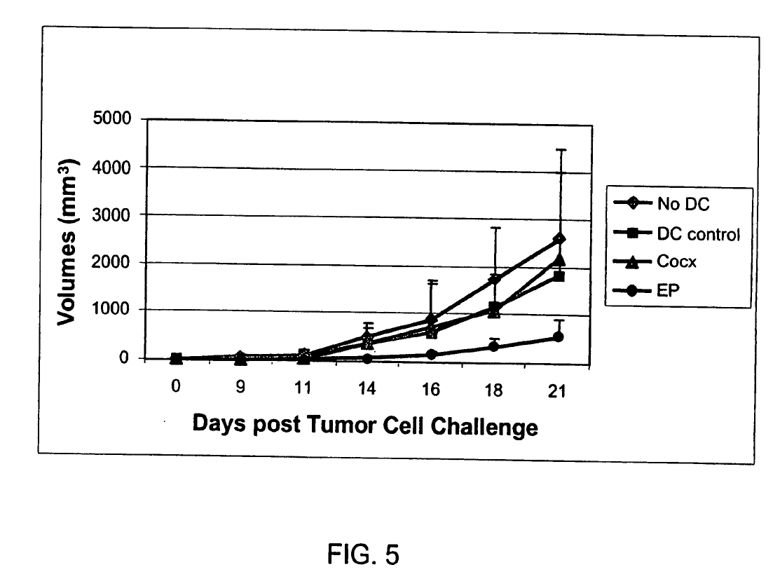

After 10 days, the mean size of tumors in mice that had received DCs that had been loaded with tumor lysate by electroporation was less than 50% than of tumors in mice that had received DCs that were loaded with tumor cell lysate by co-incubation or which received DCs that had not been loaded with tumor cell lysate or had not received any DCs. These studies demonstrated that loading of DCs by electroporation was more effective than loading by co-incubation as demonstrated by enhanced ability of such electroporation loaded DCs to augment an immune response to growing tumor cells. This decreased tumor area (or volume) continued to other days as well, not just day 10 (FIG. 5).

### Example 10

### Isolation and Stimulation of Splenocytes with DCs

DCs were isolated from C57BL6 male mice as previously described. The isolated DCs were electroporated with murine melanoma (B16-F10) lysate as previously described at the ratio at 1 tumor cell: 10 DCs. As controls, C57 mouse DCs were loaded with irrelevant or control (e.g., liver) lysate. Following electroporation, the cells were incubated for 20 minutes at 37° C. In parallel, a mixture of DCs and melanoma lysate was prepared as for electroporation, but the mixture was co-cultured for 30 minutes at 37° C. instead of being electroporated. The DCs of each mixture were transferred to 2 ml of X-VIVO 15 media supplemented with murine GM-CSF at 25 ng/mL, mouse TNF alpha at 25 ng/mL, murine interferon gamma at 25 ng/ml, lipopolysaccharide (LPS) at 5 μg/ml, and PGE (1μμg/ml). Cells were plated in low attachment plates and incubated at 37° C. overnight. The next day, the DCs were collected, counted, washed once in PBS and resuspended in X-VIVO 15 media to a concentration of 2×10⁶ cells/mL. Five hundred μL of this cell suspension of the above cells (1×10⁶ cells) were plated in 24 well low-attachment tissue culture wells. Extra DCs were cryopreserved at 2×10⁶ to 4×10⁶ cells/cryovial for restimulations.

Splenocytes were isolated from the dissected spleens of normal C57BL6 mice. The dissected spleens were washed once in PBS. The spleens were then forcefully passed through a metal mesh filter using a sterile pestle. The mesh filters were washed twice with PBS. The cell suspension was collected, centrifuged at 200×g for 10 minutes, and resuspended in 10 mL ACK red blood cell lysing solution and centrifuged again at 200×g for 10 minutes. The cells were washed once with PBS and resuspended in RPMI media supplemented with 10% FBS and plated for 2 hrs at 37° C. After 2 hrs in culture, the suspension cells were collected and counted; and any adherent cells were discarded. The splenocytes (suspension cells) were resuspended in X-VIVO 15 (Cambrex) media to a concentration of 2×10⁷ cells/ml. Five hundred μl (10×10⁶ cells) of the splenocytes solution was added to each 24 well containing DCs as described above (DCs electroporated, or co-cultured with B16 melanoma cell lysate or electroporated without any lysate). The resulting cell ratio was 1 DC: 10 splenocytes. Murine IL-2, murine IL-7 and murine GM-CSF were added to each well at a final concentration of 25 ng/ml.

Every 7 days, one vial of lysate-loaded DCs were thawed rapidly at 37° C. and resuspended in X-VIVO media. DCs were counted and resuspended at 2×10⁶ DCs/ml. Five hundred μL of each DC sample (1×10⁶ DCs) was added to the 24 wells containing DCs and splenocytes. Murine IL-2, murine IL-7 and murine GM-CSF (25 ng/ml each) were added at each restimulation. A total of 3 restimulations were performed every 7 days after the initial co-culturing of DCs and splenocytes.

Seven days after the third restimulation, splenocytes were collected from each well of a standard 24 well dish, washed in PBS and counted. Flow cytometric analysis indicated that >95% of the cells were CD3 positive T cells. The splenocytes were resuspended in RPMI media supplemented with 10% FBS at various cell concentrations.

### Example 11

### Whole Tumor Lysate Electroporated DC Induce Cytotoxic Lymphocytes and Elicit Tumor Specific Killing in vitro

Tumor cells were labeled. B16-F10 melanoma cells were collected by trypsinization, washed once in PBS and resuspended in RPMI media supplemented with 5% FBS at a final cell concentration of 1×10⁷ cells/ml. One hundred μl of B16-F10 melanoma cells (1×10⁶) were transferred to a new 1.5 ml plastic microcentrifuge tube (Eppendorf). To these cells, 100 ml of aqueous Chromium-51 (⁵¹Cr) at a stock concentration of 1 mCuries/ml was added (100 micro Curies final concentration). Cells were labeled with ⁵¹Cr for 1 hr at 37° C. ⁵¹Cr labeled cells were then washed five times with complete media and resuspended in RPMI media supplemented with 10% FBS at a final cell concentration of 1×10⁵ cells/ml.

To induce specific cell-mediated killing, 10,000 labeled tumor cells (100 μL of 1×10^{5 51}Cr-labeled B16 cells) were plated in each well of a standard U-bottomed 96 well plate. Splenocytes from above were co-cultured with the labeled tumor cells at the following ratios: 1 tumor cell: 10 (1×10⁵) splenocytes; 1 tumor cell:50 (5×10⁵) splenocytes; or 1 tumor cell:100 (1×10⁶) splenocytes. Cells co-cultured as described were incubated at 37° C. for 4 hrs. After this incubation, the 96 well plates were briefly spun at 200×g and 100 μl of the supernatant from each sample was added to scintillation vials containing 2 ml of scintillation fluid. Spontaneous release of ⁵¹Cr was determined by analyzing the supernatants of wells containing only labeled tumor cells (no splenocytes). Maximal ⁵¹Cr release from labeled tumor cells was determined by adding 100 μL of 2% Triton X-100 detergent to extra wells containing labeled tumor cells alone. Each sample was read for one minute using a scintillation counter. Results were corrected according to the following formula, wherein ER is experimental release; SR is spontaneous release; and MR is maximal release:
% Specific release=[(ER−SR)/(MR−SR)]×100.

Where experimental release (ER) is the result from each individual well of a 96 well plate. Each sample was tested in duplicate in separate wells. Statistical significance of test samples was determined by Student's paired T test.

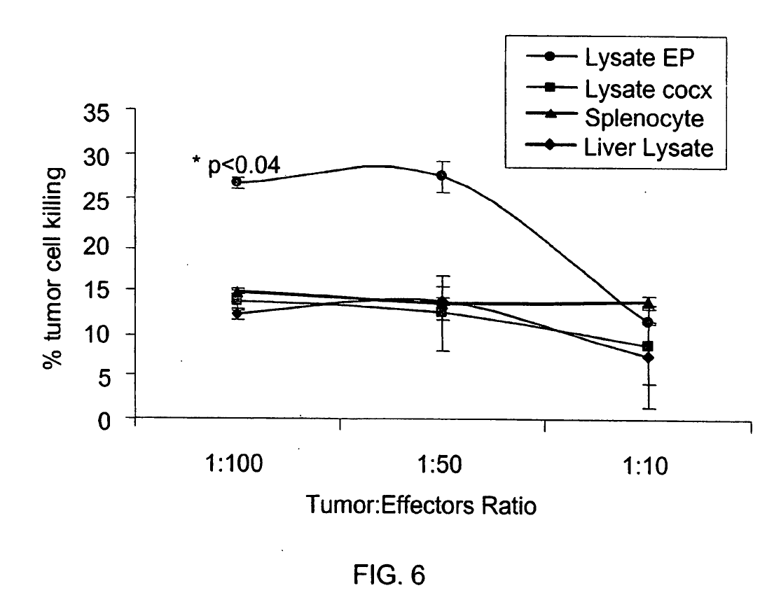

As shown in the FIG. 6, tumor killing was observed only in the electroporated DC group, but not in the co-culturing group or in the no lysate group. Previous reports showed co-culturing of tumor lysate could prime a CTL response, however, it was at a higher tumor/DC ratio. While previous reports used 1 tumor cell for each DC, or 3 tumor cells for each DC, the data presented herein demonstrated that 1 tumor cell for 10 DCs was sufficient to induce a CTL response. This amount of tumor lysate used per DC is 10 times or 30 times less tumor lysate than previously used.

### Example 12

### Whole Tumor Lysate Electroporated DC Prevent Lung Metastasis in a Therapeutic Mouse Model

C57BL6 mice were first injected intravenously (tail vein) with 5×10⁵ Lewis lung carcinoma (LLC) cells. Intravenous administration of these cells is known to elicit an aggressive tumor growth, with rapid formation of lung metastases.

DCs were isolated from C57BL6 male mice as previously described. The isolated DCs were loaded by electroporation with murine Lewis lung carcinoma (LLC) lysate as previously described for the RENCA lysate. As controls, DCs were loaded with irrelevant (whole liver) lysate. Following electroporation, the cells were incubated for 20 minutes at 37° C. In parallel a mixture of DCs and LLC lysate was prepared as for electroporation but was co-cultured for 30 minutes at 37° C. instead of being electroporated. The DCs of each mixture were transferred to 2 ml of X-VIVO 15 media supplemented with murine GM-CSF at 25 ng/ml, mouse TNF alpha at 25 ng/ml, murine interferon gamma at 25 ng/ml, lipopolysaccharide (LPS) at 5 μg/ml, and PGE (1 μg/ml). Cells were plated in low attachment plates and incubated at 37° C. overnight. The next day, the above DCs were collected, counted, washed once in PBS and resuspended in X-VIVO 15 media to a concentration of 1×10⁷ cells/ml.

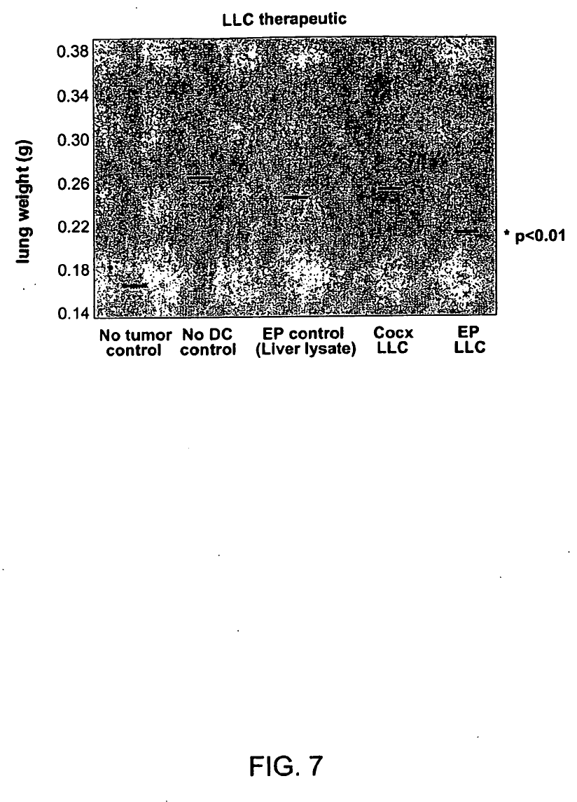

Three days after the injection of mice with LLC, 100 μl (1×10⁶) of the lysate-loaded DCs were injected into the tail veins of the mice that had received LLC. Another 3 days later (day 6), mice were given a second dose of 1×10⁶ lysate-loaded DCs. As a control, one group of mice was not given DC at all (no DC control). On day 15 post-LLC injection, mice were sacrificed and the lungs were dissected and weighed. Lung weight was used as an index of the extent of lung metastases for each group. Mice that had not been challenged with LLC were used to measure the normal (no tumor) lung weight for these mice. Administration of DCs that had been electroporated with LLC lysate caused a significant, ˜50% reduction in LLC lung metastases compared to the no DC control group (p<0.01) as shown in FIG. 7. In contrast, DCs that had been either electroporated with liver lysate or co-cultured with LLC lysate failed to have any effect upon lung metastases.

### Example 13

### Treatment of Cancer in Human Subjects Using Cancer Lysate-Loaded Human Antigen-Presenting Cells

This example describes an example of a protocol to facilitate the treatment of human cancer patients using human APCs loaded with a cancer cell lysate. In a certain embodiment, the APCs are human DCs. Patients may, but need not, have received previous chemo- radio- or gene therapeutic treatments. Optimally the patient will exhibit adequate bone marrow function (defined as peripheral absolute granulocyte count of >2,000/mm3 and platelet count of 100,000/mm³, adequate liver function (bilirubin 1.5 mg/dl) and adequate renal function (creatinine 1.5 mg/dl). One of ordinary skill in the art would understand how to isolate and load the APCs in view of this specification.

The compositions can be administered parenterally in dosage unit formulations containing standard, well known non-toxic physiologically acceptable carriers, adjuvants, and vehicles as desired. The term parenteral as used herein includes subcutaneous injections, intravenous, intramuscular, intra-arterial injection, intratumoral, or infusion techniques. The composition may be administered alone or indeed in combination with other therapies including other immunotherapies. Where a combination therapy is contemplated, the composition may be administered before, after or concurrently with the other anti-cancer agents.

In one example, a treatment course can comprise about six doses delivered over a 7 to 21 day period. Upon election by the clinician the regimen may be continued six doses every three weeks or on a less frequent (monthly, bimonthly, quarterly etc.) basis. Of course, these are only exemplary times for treatment, and the skilled practitioner will readily recognize that many other time-courses are possible.

Clinical responses can be defined by acceptable measures known to those of skill in the art. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites. Those of skill in the art will be able to take the information disclosed in this specification and optimize the treatment regimen.

### Example 14

### Clinical Trials of the Use of Cancer Lysate Loaded Human DCs in the Treatment of Cancer

This example is concerned with the development of human treatment protocols using human DCs loaded with tumor lysate in the treatment of cancer. The various elements of conducting a clinical trial, including patient treatment and monitoring, will be known to those of skill in the art in light of the present disclosure. The following information is being presented as a general guideline for use human APCs such as DCs that are loaded with cancer lysate in clinical trials pertaining to cancer treatment.

Patients with cancer chosen for clinical study will typically have failed to respond to at least one course of conventional therapy. Measurable disease is not required.

The composition may be administered alone or in combination with another chemotherapeutic agent. The administration may be intravenously such as through a catheter.

The DCs and/or anti-cancer agent combination may be administered over a short infusion time or at a steady rate of infusion over a 7 to 21 day period. The infusion may be administered alone or in combination with the anti-cancer drug. The infusion given at any dose level will be dependent upon the toxicity achieved after each. Increasing doses in combination with an anti-cancer drug will be administered to groups of patients until approximately 60% of patients show unacceptable toxicity.

Physical examination, tumor measurements, and laboratory tests should, of course, be performed before treatment and at intervals of about 3-4 weeks later. Laboratory studies should include CBC, differential and platelet count, immunological profiles, urinalysis, SMA-12-100 (liver and renal function tests), coagulation profile, and any other appropriate chemistry studies to determine the extent of disease, or determine the cause of existing symptoms. Also appropriate biological markers in serum may be monitored.

To monitor disease course and evaluate the anti-tumor responses, it is contemplated that the patients should be examined for appropriate tumor markers every 4 weeks, if initially abnormal. Laboratory studies such as a CBC, differential and platelet count, coagulation profile, and/or SMA-12-100 shall be performed weekly. Appropriate clinical studies such as radiological studies and immunological studies should be performed and repeated every 8 weeks to evaluate tumor response.

Clinical responses may be defined by acceptable measure. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,826,364
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,284,412
U.S. Pat. No. 4,366,241
U.S. Pat. No. 4,498,766
U.S. Pat. No. 4,661,913
U.S. Pat. No. 4,714,682
U.S. Pat. No. 4,767,206
U.S. Pat. No. 4,774,189
U.S. Pat. No. 4,857,451
U.S. Pat. No. 4,989,977
U.S. Pat. No. 5,160,974
U.S. Pat. No. 5,478,722
Asavaroengchai et al., Proc. Natl. Acad. Sci. USA, 99(2):931-936, 2002.
Banchereau et al., Nature, 392:245-252, 1998.
Califano et al., Cancer Res., 56(24):5720-5722, 1996.
Chang et al., Clin. Cancer Res., 8(4):1021-1032, 2002.
Campbell, In: Monoclonal Antibody Technology, Laboratory Techniques in Biochemistry and Molecular Biology, Burden and Von Knippenberg (Eds.), Elseview, Arnsterdam, 13:71-74/75-83, 1984.
De Jager et al., Semin. Nucl. Med., 23(2):165-179, 1993.
Chen et al., J. Imunol. Methods, 160(1):49-57, 1993.
Doolittle and Ben-Zeev, Methods Mol Biol, 109:215-237, 1999.
Fearon and Vogelstein, Cell, 61(5):759-767, 1990.
Gabriel and Teissié, Biophys. J., 76(4):2158-2165, 1999.
Gefter et al., Somatic Cell Genet, 3(2):231-6, 1977.
Geiger et al., Cancer Res., 61(23):8513-8519, 2001.
Goding, 1986, In: Monoclonal Antibodies: Principles and Practice, 2d ed., Academic Press, Orlando, Fla., pp. 60-61, and 71-74, 1986.
Gulbis and Galand, Hum Pathol, 24(12):1271-85, 1993.
Harding, Eur. J. Immunol., 22(7):1865-1869, 1992.
Heller et al. Tech. Canc. Res. Treat., 1(3):205-209, 2002.
Herr et al., Blood, 96(5):1857-1864, 2000.
Hibino et al., Biophys. J., 64(6):1789-1800, 1993.
Höltl et al., Clin. Cancer Res., 8(11):3369-76, 2002.
Jankowski et al., Oncogene 9:1205-11, 1994.
Kakorin et al., Biophys. Chem., 58(1-2):109-116, 1996.
Kim et al., FASEB J., 16(4):A664, 2002.
Kohler and Milstein, “Continuous cultures of fused cells secreting antibody of predefined specificity,” Nature, 256(5517):495-7, 1975.
Kohler and Milstein, “Derivation of specific antibody-producing tissue culture and tumor lines by cell fusion,” Eur J Immunol, 6(7):511-9, 1976.
Kotnik et al., Bioenerg., 43:281-291, 1998b.
Kotnik et al., Bioenerg., 45:3-16, 1998a.
Li et al., J. Leukoc. Biol., 56(5):616-624, 1994.
Mellman and Steinman, Cell, 106(3):255-258, 2001.
Mir et al., Exp. Cell Res., 175:15-25, 1988.
Mir, Bioelectrochem., 53:1-10, 2000.
Moore et al., Cell, 54:777-785, 1988.
Nakamura et al., In: Handbook of Experimental Immunology (4^{th} Ed.), Weir, Herzenberg, Blackwell, Herzenberg, (eds). Vol. 1, Chapter 27, Blackwell Scientific Publ., Oxford, 1987.
Neumann et al., Bioelectrochem Bioenerg., 48(1):3-16, 1999.
Ridge et al., Nature, 393:474-478, 1998.
Rodriguez et al., Nat. Cell Biol., 1:362-368, 1999.
Rols and Teissie, Biophys. J., 58(5):1089-1098, 1990.
Schnurr et al., Cancer Res., 62(8):2347-2352, 2002.
Scott-Taylor et al., Biochim. Biophys. Acta, 1500(3):265-279, 2000.
Van Meirvenne et al., Cancer Gene Ther., 9(9):787-797, 2002.
Van Schooten et al., J. Virol., 71(5):3776-3787, 1997.
Van Tendeloo et al., Blood, 98(1):49-56, 2001.
Weaver and Chizmadzhev, Bioenerg., 41:135-160, 1996.
Wright and Tomlinson, Immunol. Today 15:588-94, 1994.

What is claimed is:

1. A method for loading an antigen-presenting cell with one or more antigens, comprising:
a) preparing a mixture comprising antigen-presenting cells and a lysate having one or more antigens of a hyperproliferative cell, a microorganism-infected cell or a microorganism, wherein the number of antigen-presenting cells is greater than the number of cells represented in the lysate; and
b) electroporating the mixture in a manner sufficient to load the one or more antigens into the antigen-presenting cells.

2. The method of claim 1, wherein the antigen-presenting cell is a dendritic cell.

3. The method of claim 1, wherein the microorganism is a virus, bacterium, fungus, or protozoan.

4. The method of claim 1, wherein the microorganism-infected cell is a cell infected with a virus, bacterium, fungus, or protozoan.

5. The method of claim 1, wherein the lysate is prepared using a non-detergent treatment.

6. The method of claim 5, wherein the non-detergent treatment is selected from the group consisting of freeze-thaw methods, sonication methods, high pressure extrusion methods, solid shear methods, liquid shear methods, and hypotonic/hypertonic methods.

7. The method of claim 1, wherein the one or more antigens are tumor-associated antigens.

8. The method of claim 7, wherein the tumor-associated antigens are recombinant tumor-associated antigens.

9. The method of claim 1, wherein the hyperproliferative cell is a tumor cell.

10. The method of claim 9, wherein the tumor cell is an autologous tumor cell.

11. The method of claim 9, wherein the tumor cell is an allogeneic tumor cell.

12. The method of claim 9, wherein the tumor cell is further defined as a cancer cell.

13. The method of claim 12, wherein the cancer cell is a breast cancer cell, lung cancer cell, prostate cancer cell, ovarian cancer cell, brain cancer cell, liver cancer cell, cervical cancer cell, colon cancer cell, renal cancer cell, skin cancer cell, head & neck cancer cell, bone cancer cell, esophageal cancer cell, bladder cancer cell, uterine cancer cell, lymphatic cancer cell, stomach cancer cell, pancreatic cancer cell, testicular cancer cell, or leukemia cell.

14. The method of claim 3, wherein the microorganism is a virus.

15. The method of claim 3, wherein the microorganism is a bacterium.

16. The method of claim 3, wherein the microorganism is a fungus.

17. The method of claim 3, wherein the microorganism is a protozoan.

18. The method of claim 4, wherein the microorganism infected cell is a cell infected with a virus.

19. The method of claim 4, wherein the microorganism infected cell is a cell infected with a bacterium.

20. The method of claim 4, wherein the microorganism infected cell is a cell infected with a fungus.

21. The method of claim 4, wherein the microorganism infected cell is a cell infected with a protozoan.

22. The method of claim 1, wherein the lysate is prepared using a detergent.

23. The method of claim 13, wherein the cancer cell is a renal cancer cell.

24. The method of claim 13, wherein the cancer cell is a skin cancer cell.

25. The method of claim 13, wherein the cancer cell is a lung cancer cell.

26. The method of claim 13, wherein the cancer cell is a breast cancer cell.

27. The method of claim 13, wherein the cancer cell is a leukemia cell.

28. The method of claim 13, wherein the cancer cell is a prostate cancer cell.

29. The method of claim 1, wherein the number of antigen-presenting cells to the number of cells represented in the lysate is between about 2:1 to 150:1.

30. The method of claim 1, wherein the number of antigen-presenting cells to the number of cells represented in the lysate is between about 5:1 to 100:1.

31. The method of claim 1, wherein electroporating the mixture comprises static electroporation.

32. The method of claim 1, wherein electroporating the mixture comprises flow electroporation.

33. A method of treating or preventing a disease in a subject, comprising:
a) loading an antigen-presenting cell with a lysate having one or more antigens of a hyperproliferative cell, a microorganism, or a microorganism-infected cell using electroporation;
b) preparing a composition of said antigen-presenting cell; and
c) administering to a subject in need thereof an effective amount of said composition.

34. The method of claim 33, further comprising culturing the antigen-presenting cell.

35. The method of claim 33, wherein the subject is a mammal.

36. The method of claim 33, wherein the subject is a human.

37. The method of claim 33, wherein the disease is a hyperproliferative disease.

38. The method of claim 37, wherein the hyperproliferative disease is a tumor.

39. The method of claim 38, wherein the tumor is a cancer.

40. The method of claim 39, wherein the cancer is breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, or leukemia.

41. The method of claim 33, wherein the subject is undergoing secondary anti-hyperplastic therapy.

42. The method of claim 41, wherein the secondary anti-hyperplastic therapy is chemotherapy, radiotherapy, immunotherapy, phototherapy, cryotherapy, toxin therapy, hormonal therapy, or surgery.

43. The method of claim 33, wherein the composition is delivered systemically, intravascularly, intradermally, or subcutaneously.

44. The method of claim 33, wherein the composition is delivered locally to a tumor mass.

45. The method of claim 33, wherein the antigen-presenting cells comprise dendritic cells.
